(19) United States
(12) Patent Application Publication
(10) Pub. No.: US 2024/0324953 A1
(43) Pub. Date: Oct. 3, 2024

(54) OPIOID OVERDOSE DETECTION USING PATTERN RECOGNITION

(71) Applicant: MASIMO CORPORATION, Irvine, CA (US)

(72) Inventors: Valery G. Telfort, Irvine, CA (US); Kostantinos Michalopoulos, Irvine, CA (US); Jerome J. Novak, JR., Lake Forest, CA (US); Ammar Al-Ali, San Juan Capistrano, CA (US)

(21) Appl. No.: 18/625,126

(22) Filed: Apr. 2, 2024

Related U.S. Application Data
(60) Provisional application No. 63/493,981, filed on Apr. 3, 2023.

(51) Int. Cl. A61B 5/00; A61B 5/0205; A61B 5/026; A61B 5/08; A61B 5/1455

(52) U.S. Cl. CPC A61B 5/4845; A61B 5/02055; A61B 5/14552; A61B 5/7275; A61B 5/7282; A61B 5/7425; A61B 5/746; A61B 5/747; A61B 5/0261; A61B 5/0816; A61B 2562/0238

(57) ABSTRACT

An opioid overdose monitoring system configured to generate an overdose risk score of a user of a wearable device can include a physiological sensor coupled to the wearable device having one or more light emitting diodes of the physiological sensor configured to transmit an optical radiation into the tissue site of the user. One or more detectors of the physiological sensor can respond to an intensity of the optical radiation after absorption by the tissue site of the user. At least one hardware processor in communication with the physiological sensor can determine a plurality of parameters based at least on the attenuated light, determine a plurality of characteristics based on the plurality of parameters associated with at least one of instantaneous values and historical physiological parameter, determining an overdose risk score, determine an alarm level, and implement an intervention associated with the determined alarm level.

### RELATED APPLICATIONS

This application claims priority to U.S. Provisional Application No. 63/493,981, filed Apr. 3, 2023, titled “Opioid Overdose Detection Using Pattern Recognition”. This application is related to U.S. application Ser. No. 16/432,739, filed on Jun. 5, 2019 and titled “Opioid Overdose Monitoring,” U.S. application Ser. No. 16/432,756 filed on Jun. 5, 2019 and titled “Opioid Overdose Monitoring,” U.S. application Ser. No. 16/432,703 filed on Jun. 5, 2019 and titled “Opioid Overdose Monitoring,” U.S. application Ser. No. 17/145,663 filed on Jan. 11, 2021 and titled “Opioid Overdose Monitoring,” U.S. application Ser. No. 16/928,531 filed on Jul. 14, 2020 and titled “Locating a Locally Stored Medication,” U.S. application Ser. No. 17/116,155 filed on Dec. 9, 2020 and titled “Kit of Opioid Overdose Monitoring,” U.S. application Ser. No. 17/830,263 filed on Jun. 1, 2022 and titled “Time-Based Critical Opioid Blood Oxygen Monitoring,” and U.S. application Ser. No. 18/045,120 filed Oct. 7, 2022 and titled “Opioid Overdose Detection Using Pattern Recognition,” which are incorporated by reference herein in their entirety. All of the above-listed applications and any and all applications for which a foreign or domestic priority claim is identified in the Application Data Sheet as filed with the present application are hereby incorporated by reference under 37 CFR 1.57.

### FIELD

The present disclosure relates generally to the field of detecting an opioid overdose, and in particular, to detecting low saturation of oxygen in the blood of an opioid user, and automatically notifying a responder.

### BACKGROUND

Substance abuse disorders impact the lives of millions of people. An opioid overdose can occur when a person overdoses on an illicit opioid drug, such as heroin or morphine. Many controlled substances are prescribed by physicians for medical use. Patients can accidentally take an extra dose or deliberately misuse a prescription opioid. Mixing a prescription opioid with other prescription drugs, alcohol, or over-the-counter-medications can cause an overdose. Children are particularly susceptible to accidental overdoses if they take medication that is not intended for them. Opioid overdose is life-threatening and requires immediate emergency attention.

### SUMMARY

An opioid overdose is toxicity due to an excess or opioids. Symptoms of an opioid overdose include marked confusion, delirium, or acting drunk; frequent vomiting; pinpoint pupils; extreme sleepiness, or the inability to wake up; intermittent loss of consciousness; breathing problems, including slowed or irregular breathing; respiratory arrest (absence of breathing); respiratory depression (a breathing disorder characterized by slow and ineffective breathing); and cold, clammy skin, or bluish skin around the lips or under the fingernails.

Depressed breathing is the most dangerous side effect of opioid overdose. Lack of oxygen to the brain can not only result in permanent neurologic damage, but may also be accompanied by the widespread failure of other organ systems, including the heart and kidneys. If a person experiencing an opioid overdose is left alone and asleep, the person could easily die as their respiratory depression worsens.

Oximetry can be used to detect depressed breathing. Oximetry utilizes a noninvasive optical sensor to measure physiological parameters of a person. In general, the sensor has light emitting diodes (LEDs) that transmit optical radiation into a tissue site and a detector that responds to the intensity of the optical radiation after absorption (e.g., by transmission or transflectance) by, for example, pulsatile arterial blood flowing within the tissue site. Based on this response, a processor can determine measurements for peripheral oxygen saturation (SpO₂), which is an estimate of the percentage of oxygen bound to hemoglobin in the blood, pulse rate, plethysmograph waveforms, which indicate changes in the volume of arterial blood with each pulse beat, and perfusion quality index (e.g., an index that quantifies pulse strength at the sensor site), among many others.

It is noted that “oximetry” as used herein encompasses its broad ordinary meaning known to one of skill in the art, which includes at least those noninvasive procedures for measuring parameters of circulating blood through spectroscopy. Moreover, “plethysmograph” as used herein (commonly referred to as “photoplethysmograph”), encompasses its broad ordinary meaning known to one of skill in the art, which includes at least data representative of a change in the absorption of particular wavelengths of light as a function of the changes in body tissue resulting from pulsing blood. An oximeter that is compatible with a hand held monitor, such as a mobile computing device, can be used to monitor physiological parameters. The oximeter can detect decreased oxygen saturation in the blood of the user. Decreased oxygen saturation in the blood of the user is an indication of respiratory distress, which can be an indication of opioid overdose. Once the oxygen saturation of the user falls below an acceptable threshold, a software application in the mobile computing device can alert others to provide emergency help. The threshold can be set to provide an early indication of an overdose event. If the overdose is caught early, emergency treatment can be provided before irreparable harm occurs.

In some implementations, an opioid overdose monitoring system is configured to generate an overdose risk score of a user of a wearable device: a physiological sensor coupled to the wearable device, said physiological sensor configured to detect attenuated light from a tissue site of the user; one or more light emitting diodes of the physiological sensor configured to transmit an optical radiation into the tissue site of the user; one or more detectors of the physiological sensor configured to respond to an intensity of the optical radiation after absorption by the tissue site of the user; a display configured to display one or more screens; and at least one hardware processor in communication with the physiological sensor, the at least one hardware processor configured to: determine a plurality of parameters based at least on the attenuated light from the physiological sensor, the plurality of parameters associated with at least two branches of physiology; determine a plurality of characteristics based on the plurality of parameters, the plurality of characteristics associated with at least one of instantaneous values and historical physiological parameter data of the plurality of parameters, the plurality of characteristics including user trend characteristics and stability characteristics, wherein the user trend characteristics track the plurality of parameters over a period of time, and wherein the stability characteristics measure a degree of which instantaneous parameter values corresponding to plurality of parameters tend to deviate from their baseline, wherein large and frequent deviations are indicative of high parameter instability; determining an overdose risk score based on at least the user trend characteristics and the stability characteristics; determine an alarm level of a series of escalating alarm levels based on the overdose risk score; and implement an intervention associated with the determined alarm level.

In some implementations, the plurality of parameters includes at least one of oxygen saturation (SpO₂), respiration (PR), and perfusion index (PI). In some implementations, the plurality of parameters further includes at one of respiration rate from the pleth (RRp) and temperature. In some implementations, the at least one hardware processor monitors a lower limit of the SpO₂. In some implementations, the lower limit is set by at least one of a user and care provider. In some implementations, the lower limit is set based on the plurality of parameters. In some implementations, the lower limit is 85.

In some implementations, the at least one hardware processor is further configured to, for each of the plurality of parameters, determine a baseline risk, an instability index, an average slope, and desaturation pressure, and determine a weighted aggregate of the baseline risk, the instability index, the average slope, and the desaturation pressure. In some implementations, the alarm level is characterized by values of the overdose risk score, a normalized area corresponding to SpO₂ levels over a period of time, and SpO₂. In some implementations, the overdose risk score is at least based on a history of the plurality of parameter. In some implementations, the physiological sensor detects the plurality of parameters periodically. In some implementations, the period of sensing is every second.

In some implementations, the at least one hardware processor is further configured to determine unavailability or unreliability of the plurality of parameters. In some implementations, the at least one hardware processor is furthered configured to correlate one or more trends of the plurality of parameters. In some implementations, the at least one processor further correlates the trends of multiple physiological parameters. In some implementations, the at least one hardware processor is configured to determine a plurality of alarm levels in parallel. In some implementations, the at least one hardware processor is further configured to determines a presence of an event based on a crossing of at least one of a first and instantaneous baseline across one or more event thresholds.

In some implementations, the intervention associated with the determined alarm level indicates a local rescue. In some implementations, the local rescue generates an audible alarm. In some implementations, the intervention associated with the determined alarm level initiates an intermediate rescue. In some implementations, the intermediate rescue transmits wirelessly a notification to one or more recipients. In some implementations, the intermediate rescue stimulates the user physically. In some implementations, the intervention associated with the determined alarm level initiates professional assistance. In some implementations, the professional assistance notifies medical personnel to respond with an opioid receptor antagonist.

In some implementations, the at least one processor is further configured to output an indicator flag that the overdose risk score is valid. In some implementations, the alarm level is characterized by values of the overdose risk score, a normalized area, and a physiological parameter.

In some implementation, an opioid overdose monitoring system configured to generate an overdose risk score of a user can include: a physiological sensor, said physiological sensor configured to detect attenuated light from a tissue site of the user; at least one hardware processor in communication with the physiological sensor, the at least one hardware processor configured to: one or more light emitting diodes of the physiological sensor configured to transmit an optical radiation into the tissue site of the user; one or more detectors of the physiological sensor configured to respond to an intensity of the optical radiation after absorption by the tissue site of the user; a display configured to display one or more screens; and at least one hardware processor in communication with the physiological sensor, the at least one hardware processor configured to: determine a plurality of parameters based on the attenuated light from the physiological sensor, the plurality of parameters associated with at least two branches of physiology; determine a plurality of characteristics based on the plurality of parameters, the plurality of characteristics associated with at least one of instantaneous values and historical physiological parameter data of the plurality of parameters, the plurality of characteristics including user trend characteristics and stability characteristics, wherein the user trend characteristics track the plurality of parameters over a period of time, and wherein the stability characteristics measure a degree of which instantaneous parameter values corresponding to plurality of parameters tend to deviate from their baseline, where large and frequent deviations are indicative of high parameter instability; determining an overdose risk score based on at least the user trend characteristics and the stability characteristics; determine an alarm level of a series of escalating alarm levels based on the overdose risk score; and implement an intervention associated with the determined alarm level.

In some implementations, the plurality of parameters includes at least one of oxygen saturation (SpO₂), respiration (PR), and perfusion index (PI). In some implementations, the plurality of parameters further includes at one of respiration rate from the pleth (RRp) and temperature. In some implementations, the at least one hardware processor monitors a lower limit of the SpO₂. In some implementations, the lower limit is set by at least one of a user and care provider. In some implementations, the lower limit is set based on the plurality of parameters. In some implementations, the lower limit is 85.

In some implementations, the at least one hardware processor is further configured to, for each of the plurality of parameters, determine a baseline risk, an instability index, an average slope, and desaturation pressure, and determine a weighted aggregate of the baseline risk, the instability index, the average slope, and the desaturation pressure. In some implementations, the alarm level is characterized by values of the overdose risk score, a normalized area corresponding to SpO₂ levels over a period of time, and SpO₂.

In some implementations, the overdose risk score is at least based on a history of the plurality of parameter. In some implementations, the physiological sensor detects the plurality of parameters periodically. In some implementations, the period of sensing is every second.

In some implementations, the at least one hardware processor is further configured to determine unavailability or unreliability of the plurality of parameters. In some implementations, the at least one hardware processor is furthered configured to correlate one or more trends of the plurality of parameters. In some implementations, the at least one processor further correlates the trends of multiple physiological parameters. In some implementations, the at least one hardware processor is further configured to output an indicator flag that the overdose risk score is valid.

In some implementations, the at least one hardware processor is further configured to determines a presence of an event based on a crossing of at least one of a first and instantaneous baseline across one or more event thresholds. In some implementations, the at least one hardware processor is configured to determine a plurality of alarm levels in parallel.

In some implementations, the intervention associated with the determined alarm level indicates a local rescue. In some implementations, the local rescue generates an audible alarm. In some implementations, the intervention associated with the determined alarm level initiates an intermediate rescue. In some implementations, the intermediate rescue includes at least one of transmitting wirelessly a notification to one or more recipients and stimulating the user physically. In some implementations, the intervention associated with the determined alarm level initiates a professional assistance. In some implementations, the professional assistance notifies medical personnel to respond with an opioid receptor antagonist. In some implementations, the alarm level is characterized by values of the overdose risk score, a normalized area, and a physiological parameter.

For purposes of summarizing the disclosure, certain aspects, advantages, and novel features are discussed herein. It is to be understood that not necessarily all such aspects, advantages or features will be embodied in any particular embodiment of the invention, and an artisan would recognize from the disclosure herein a myriad of combinations of such aspects, advantages, or features.

### BRIEF DESCRIPTION OF THE DRAWINGS

Various embodiments will be described hereinafter with reference to the accompanying drawings. The drawings and the associated descriptions are provided to illustrate embodiments of the present disclosure and do not limit the scope of the claims. In the drawings, similar elements have similar reference numerals.

FIG. 1A is an overview of an example opioid use monitoring system.

FIG. 1B is a diagrammatic representation of an example network associated with monitoring opioid.

FIG. 1C is an overview of another example opioid use monitoring system.

FIG. 2A is a block diagram of an example physiological monitoring system.

FIG. 2B is a flow chart of an example process to monitor physiological parameters for opioid use and provide notifications.

FIGS. 3A-3E illustrate various example software applications to provide information, notifications, and alerts to opioid users, first responders, medical personnel, and friends.

FIG. 4 is a flow chart of an example process to monitor for opioid overdose.

FIGS. 5A-5F illustrate various example software applications to trigger an alarm and notify a friend when an opioid overdose is indicated.

FIGS. 6A-6L illustrate various examples of physiological parameter sensors and signal processing devices.

FIG. 6M illustrates an example fingertip sensor that can be coupled to a wearable device.

FIG. 6N illustrates an example embodiment of a wearable device with a display screen.

FIG. 6O illustrates a top view of an example embodiment of a physiological parameter measurement sensor or module.

FIG. 7A is a block diagram of an example opioid user system environment and an example cloud environment.

FIG. 7B is a block diagram illustrating example components of a cloud environment.

FIG. 7C is a block diagram illustrating example components of an opioid user system of an example opioid user system environment.

FIG. 8 is a flowchart of an example process to notify an opioid user's notification network of the status of the opioid user.

FIG. 9A is a block diagram of an example opioid use monitoring system.

FIGS. 9A1-9A13 illustrate various example software applications to trigger an alarm and notify a friend when an opioid overdose is indicated.

FIG. 9B is a flow diagram of an example process to administer the opioid receptor antagonist using the system of FIG. 9A.

FIG. 10 is an example of a medical monitoring hub device used on the opioid use monitoring system of FIG. 9A.

FIG. 11 illustrates a block diagram of an example risk score determination process for measured physiological parameters.

FIG. 12 illustrates a block diagram of an example alarm level determination process for an example opioid overdose risk determination.

FIG. 13 illustrates example physiological data associated with an example opioid user's session.

FIG. 14 illustrates a block diagram of example processing that may be performed on measured physiological parameters.

FIG. 15 illustrates a block diagram of example processing that may be performed to determine characteristics of a measured peripheral oxygen saturation (SpO₂).

FIG. 16 illustrates a block diagram of example processing that may be performed to determine characteristics of a measured Pulse Rate (PR).

FIG. 17 illustrates a block diagram of example processing that may be performed to determine characteristics of a measured Perfusion Index (PI).

FIG. 18 illustrates another example physiological data associated with an example opioid user's session.

### DETAILED DESCRIPTION

Although certain embodiments and examples are described below, this disclosure extends beyond the specifically disclosed embodiments and/or uses and obvious modifications and equivalents thereof. Thus, it is intended that the scope of this disclosure should not be limited by any particular embodiments described below.

### Overview

An application for a mobile computing device that is used in conjunction with a physiological parameter monitoring assembly to detect physiological parameters of an opioid user can comprise determining a physiological condition of the opioid user based at least in part on the physiological parameters, and providing notifications based at least in part on the physiological condition of the opioid user. The physiological parameter monitoring assembly can be a pulse oximeter that includes a sensor and a signal processing device. Examples of physiological parameters that can be monitored are peripheral oxygen saturation (SpO₂), respiration, and perfusion index (PI). The application can determine the physiological condition of the user based on the SpO₂ alone, respiration alone, PI alone, a combination of the SpO₂ and respiration, a combination of the SpO₂ and PI, a combination of the respiration and the PI, or a combination of the SpO₂, respiration, and PI.

The application can request user input and determine the physiological condition of the user based at least in part on the received user input and the physiological parameters from the pulse oximeter. The determination of the user's condition can be based on the user input and one or more of peripheral oxygen saturation (SpO₂), respiration, and perfusion index (PI). The application can learn, based at least in part on stored physiological parameters, trends in user's the physiological reaction to opioid use to better anticipate overdose events of the user.

The application can notify one or more of caregivers, loved ones, friends, and first responders of an overdose event. The application can provide “everything OK” notifications upon request or periodically to concerned family and friends. The application can provide detailed care instructions to first responders. The application can provide the location of the user, the location of the closest medication to reverse the effects of an opioid overdose, or the location of the closest medical personnel. The application can provide one or more of visual, audible, and sensory (vibration) alerts to the user with increasing frequency and intensity to the user.

An application for a mobile computing device that is used in conjunction with a sensor and a signal processing device to detect abnormally low blood oxygen saturation that is indicative of an overdose event in a user can comprise triggering an alarm, and notifying others of the overdose event. This increases the likelihood that opioid users, their immediate personal networks, and first responders are able to identify and react to an overdose by administrating medication to reverse the effects of the overdose. Such medication can be considered an opioid receptor antagonist or a partial inverse agonist. Naloxone or Narcan® is a medication that reverses the effect of an opioid overdose and is an opioid receptor antagonist. Buprenorphine or Subutex® is an opioid used to treat opioid addiction. Buprenorphine combined with naloxone or Suboxone® is a medication that may also be used to reverse the effect of an opioid overdose. Other example medications are naltrexone, nalorphine, and levallorphan. Administration can be accomplished by intravenous injection, intramuscular injection, and intranasally, where a liquid form of the medication is sprayed into the user's nostrils. Administration of the medication can also occur via an endotracheal tube, sublingually, where a gel or tablet of the medication is applied under the tongue, and transdermally, where the medication can be a gel applied directly to the skin or within a transdermal patch applied to the skin.

A system to monitor a user for an opioid overdose condition can comprise a sensor configured to monitor one or more physiological parameters of a user, a signal processing device configured to receive raw data representing the monitored one or more physiological parameters and to provide filtered parameter data; and a mobile computing device configured to receive the one or more physiological parameters from the signal processing device. The mobile computing device comprises a user interface, a display, network connectivity, memory storing an application as executable code, and one or more hardware processors. The application monitors the physiological parameters to determine a condition of the user and provides notifications to the user, to a crowd-sourced community of friends, family, and other opioid users that have also downloaded the application onto their computing devices, and to emergency providers and medical care personnel.

Home pulse oximetry monitoring systems for opioid users can include a pulse oximeter, such as a Masimo Rad-97 Pulse CO-Oximeter®, for example, and sensors, such as Masimo LNCS® adhesive sensors and the like, to detect blood oxygen levels and provide alerts and alarms when the opioid user's blood oxygen level drops below a threshold. The home monitoring system can provide alarm notifications that can alert a family member, remote caregiver, and a first responder, for example, to awaken the opioid user and to administer the antidote for an opioid overdose, such as an opioid receptor antagonist.

The mobile computing device can be configured to receive the filtered parameter data from the signal processing device; display representations of the filtered parameter data on the display, where the filtered parameter data includes at least oxygen saturation data for the oxygen level in the blood of the user; compare a current oxygen saturation value to a minimum oxygen saturation level; trigger an alarm when the current oxygen saturation value is below the minimum oxygen saturation level; and provide notifications over a network to another when the current oxygen saturation value is below the minimum oxygen saturation level.

The display can display the representations of the filtered parameter data as dials indicating acceptable and acceptable ranges. The filtered parameter data can include one or more of heart rate data, respiration rate data, pleth variability data, perfusion index data, and respiratory effort index data. The application can provide notifications to the user and can provide notifications to others. The notification can be one or more of a text message, an email, and a phone call. The notification can include a current value of oxygen saturation and a graph indicting a trend of the oxygen saturation levels. The notification can further include one or more of a phone number of the user, a location of the user, directions to the location of the user, a closest location of naloxone or other medication used to reverse the effects of an opioid overdose. The notification can be an automatic call to emergency responders.

A system to monitor a user for an opioid overdose condition can comprise one or more computing devices associated with an opioid overdose monitoring service. The opioid overdose monitoring service can be configured to identify opioid monitoring information from at least one physiological monitoring system associated with a user, where the opioid monitoring information comprises one of an overdose alert and a non-distress status, retrieve over a network notification information associated with the user, where the notification information includes first contact information associated with the overdose alert and second contact information associated with the non-distress status, send an overdose notification using the first contact information in response to the opioid monitoring information that indicates the overdose alert, and send a non-distress notification using the second contact information in response to the opioid monitoring information that indicates the non-distress status.

The system can further comprise a physiological monitoring system comprising a sensor configured to monitor one or more physiological parameters of the user and a signal processing board configured to receive raw data representing the monitored one or more physiological parameters and to provide filtered parameter data, and a mobile computing device comprising a display, network connectivity, memory storing executable code, and one or more hardware processors. The mobile computing device can be configured to receive the filtered parameter data from the signal processing board, display representations of the filtered parameter data on the display, where the filtered parameter data includes at least oxygen saturation data for the oxygen level in the blood of the user, compare a current oxygen saturation value to a minimum oxygen saturation level, and trigger an alarm when the current oxygen saturation value is below the minimum oxygen saturation level.

The mobile computing device can be configured to receive the filtered parameter data from the signal processing board, generate the opioid monitoring information based on the filtered parameter data, and send the opioid monitoring information over a network to the opioid overdose monitoring service. The filtered parameter data can include one or more of a current oxygen saturation value, heart rate data, respiration rate data, pleth variability data, perfusion index data, and respiratory effort index data. The overdose and non-distress notifications can comprise one or more of a text message, an email, and a phone call. The overdose and non-distress notifications can include a current value of oxygen saturation and a graph indicting a trend of the oxygen saturation levels. The overdose notification can comprise one or more of a phone number of the user, a location of the user, directions to the location of the user, a closest location of naloxone or other medication used to reverse the effects of an opioid overdose. The overdose notification can automatically calls emergency responders. The network can be the Internet.

A kit for monitoring for an opioid overdose event can comprise a sensor to sensor physiological parameters and a medical monitoring hub device to receive indications of the sensed physiological parameters and to receive an indication of an opioid overdose event. The kit can further comprise a delivery device to deliver medication in response to the indication of the opioid overdose event. The delivery device can automatically administer an opioid receptor antagonist in response to the indication of an opioid overdose event. The delivery device can comprise a patch that includes a reservoir with the medication, a needle, and a battery. The hub device can comprise memory for storage of the indication of the sensed physiological parameters. The hub device can receive and store data from monitoring devices other than the sensor. The data from the monitoring devices can comprise data associated with a well-being of a user. The kit may be available without a prescription.

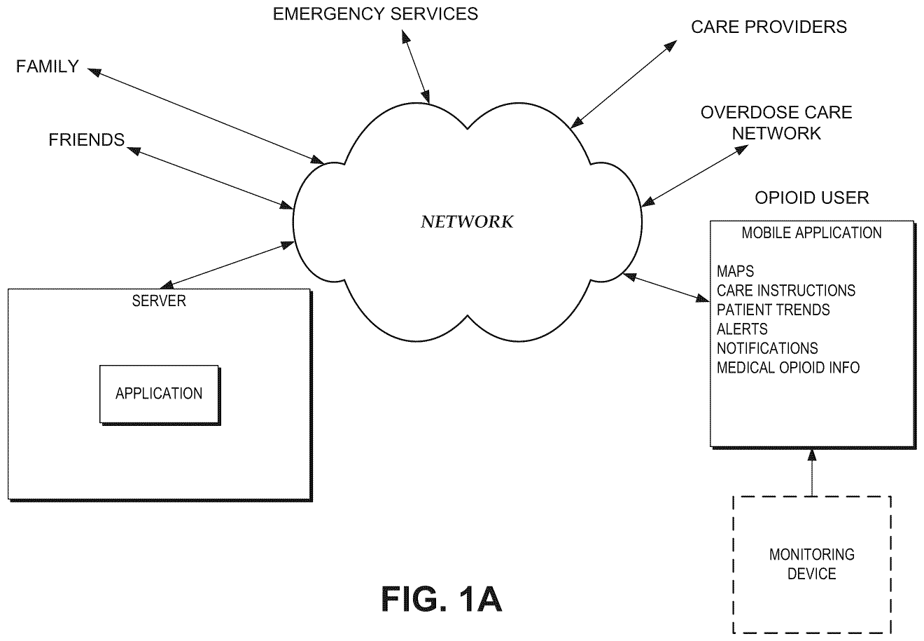

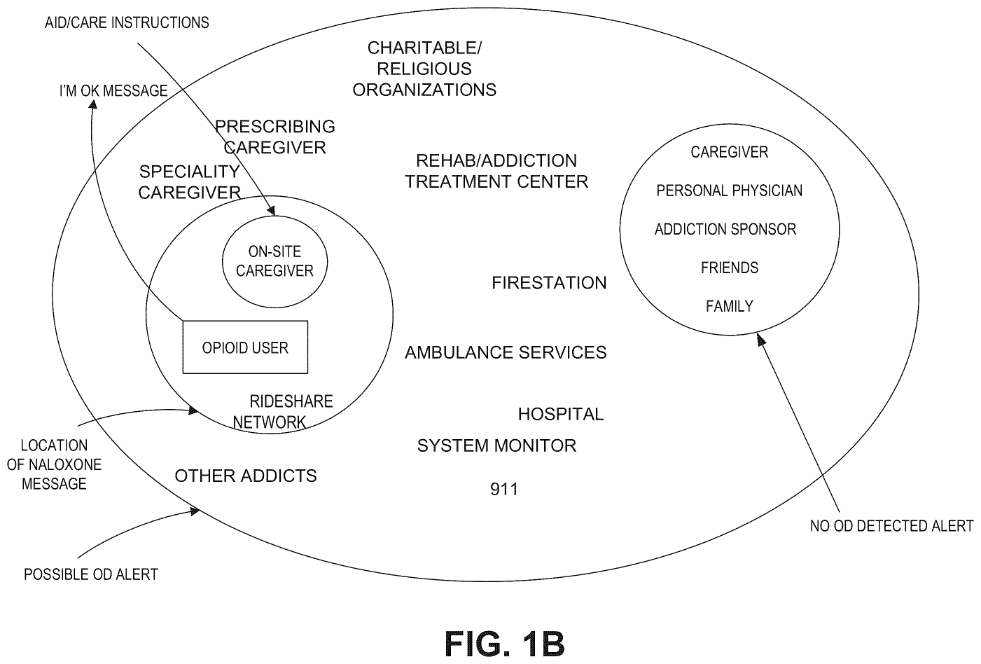

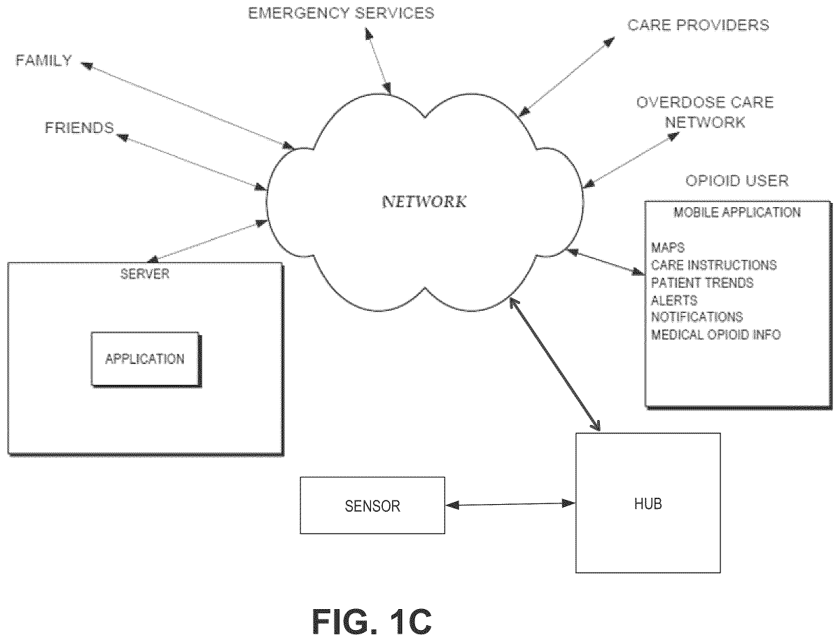

FIG. 1A is an overview of an example opioid use monitoring/notification system. The opioid users' support network can include friends, family, emergency services, care providers, and overdose care networks, for example that communicate over a network, such as the Internet. The support network receives notifications and/or status updates of the opioid user's condition. An optional monitoring device can monitor the opioid user's respiration and other biological parameters, such as heart rate, blood oxygen saturation, perfusion index, for example, and provides the parameters to the smart device. An application running on the smart device can determine whether an opioid overdose event is imminent and/or occurring. The application can also provide additional information, such as care instructions, patient trends, medical opioid information, care instruction, user location, the location of naloxone, buprenorphine, buprenorphine in combination with naloxone, or other medication used to reverse the effects of an opioid overdose, and the like. The support network, after receiving a notification, can communicate with a central server to obtain the additional information.

FIG. 1B is a diagrammatic representation of an example support network associated with monitoring opioid use. The diagram illustrates an example of an opioid use support network. An opioid user may want to notify friends, family, and caregivers when they are in need of emergency care due to indications that an opioid overdose is imminent or occurring. The diagram illustrates an example of an opioid use support network. Subnetworks within the support network may receive different notifications. For example, caregivers, such as emergency 911 services, rideshare services, such as Uber® and Lyft®, for example, treatment centers, prescribing caregivers, specialty caregivers, ambulance services can receive possible overdose alerts in order to provide the immediate life-saving care to the user; an on-site caregiver can receive care instructions; friends and family can receive periodic status messages indicating no overdose event occurring; and transportation services can receive messages with the location of medications used to reverse the effects of an opioid overdose, such as naloxone, buprenorphine, a combination of buprenorphine and naloxone, and the like. Other subnetworks receiving different notifications are possible.

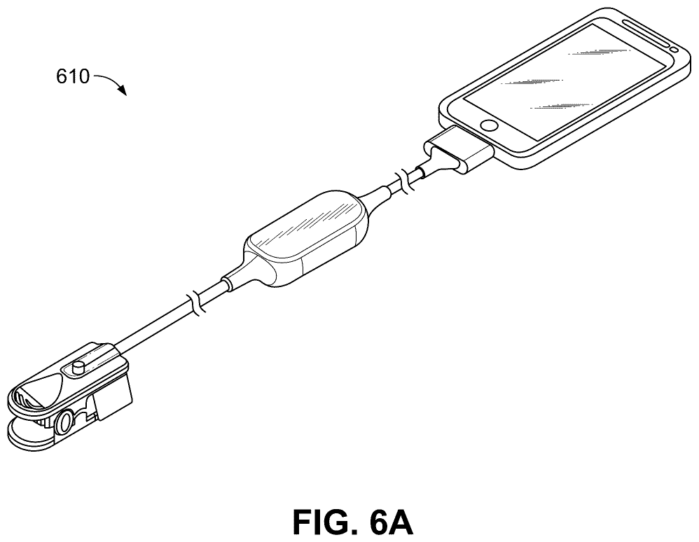

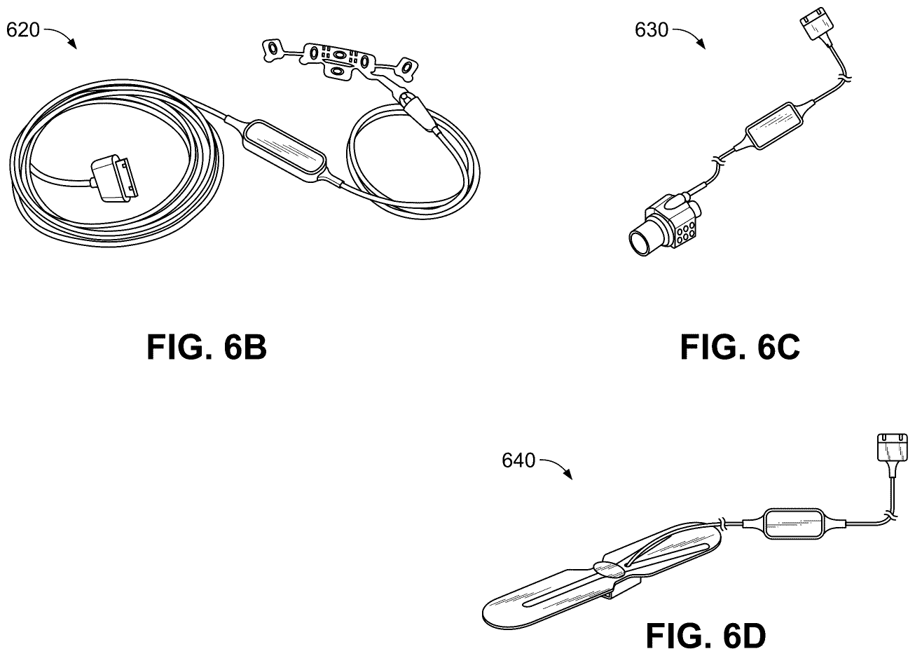

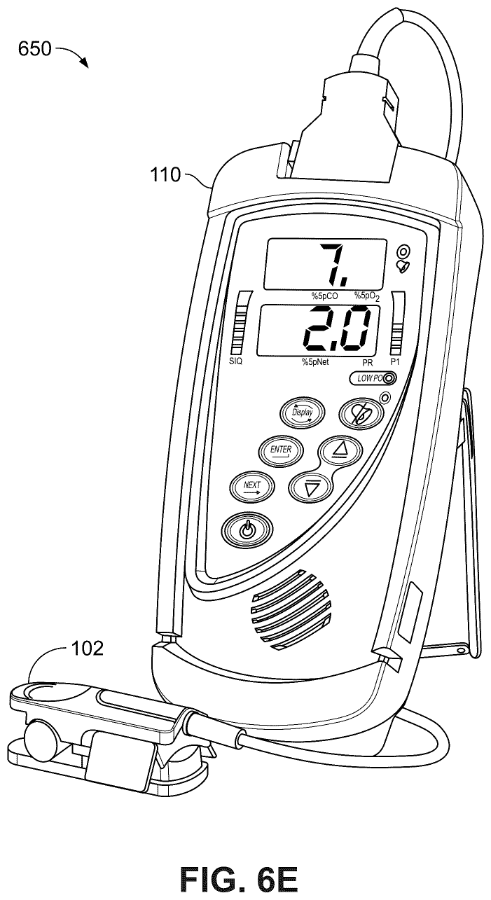

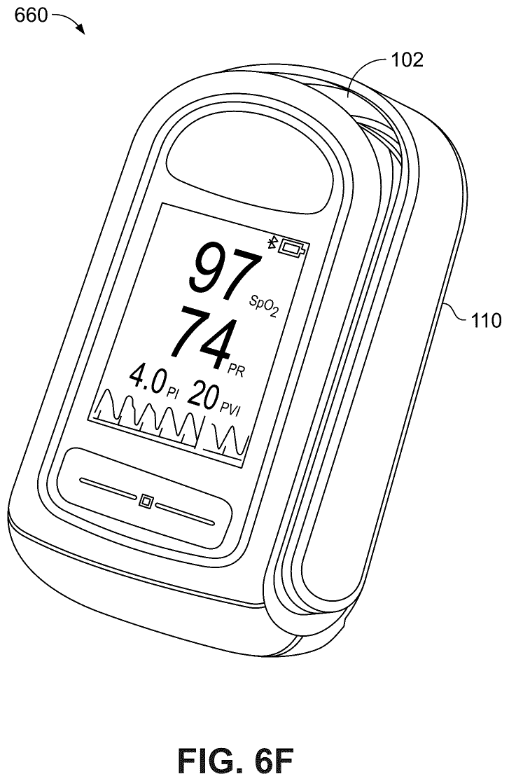

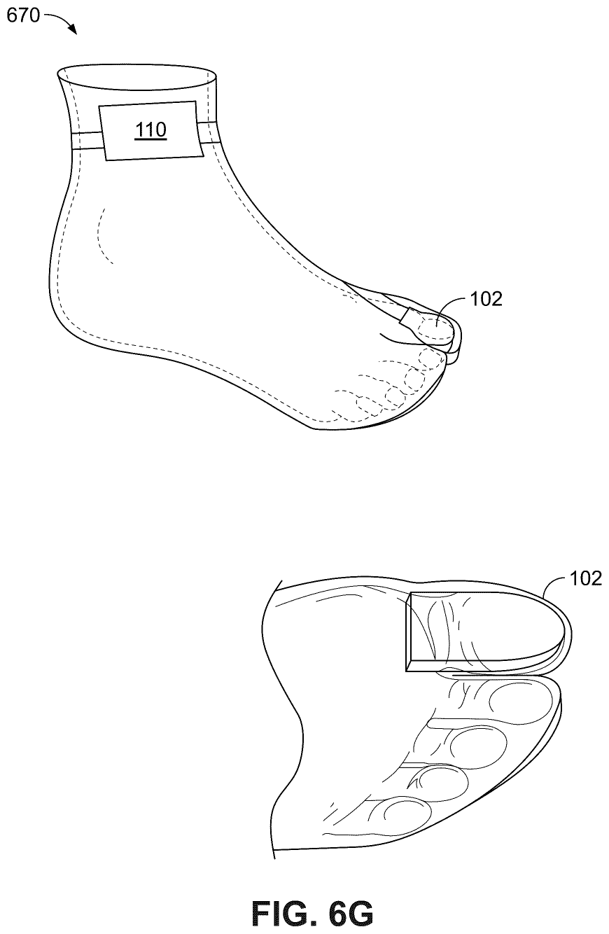

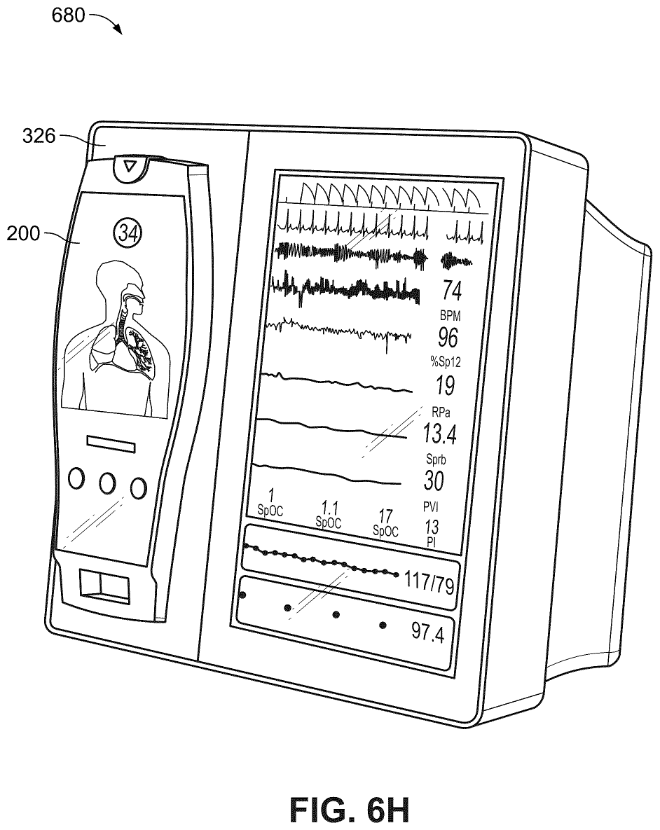

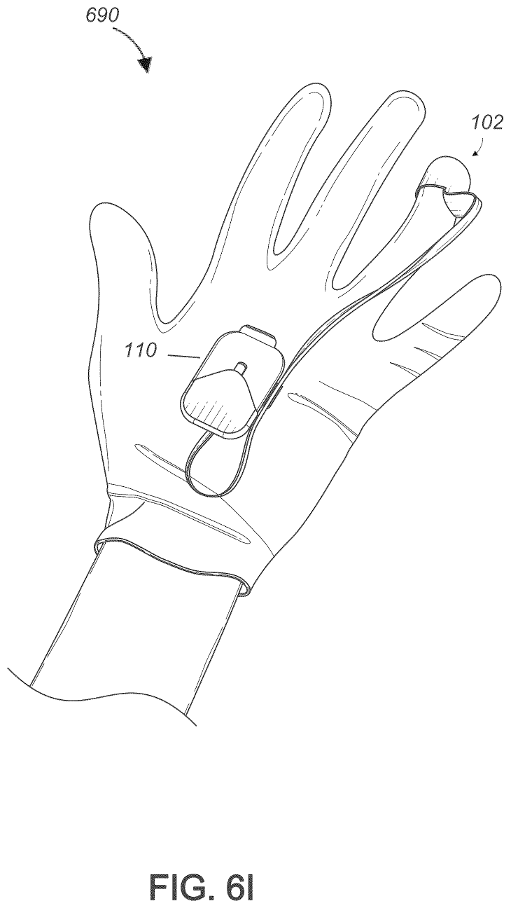

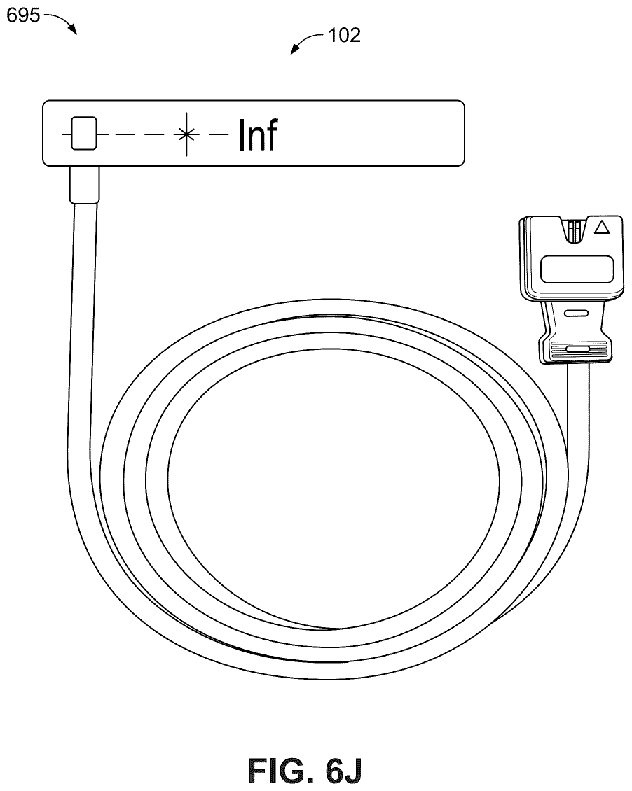

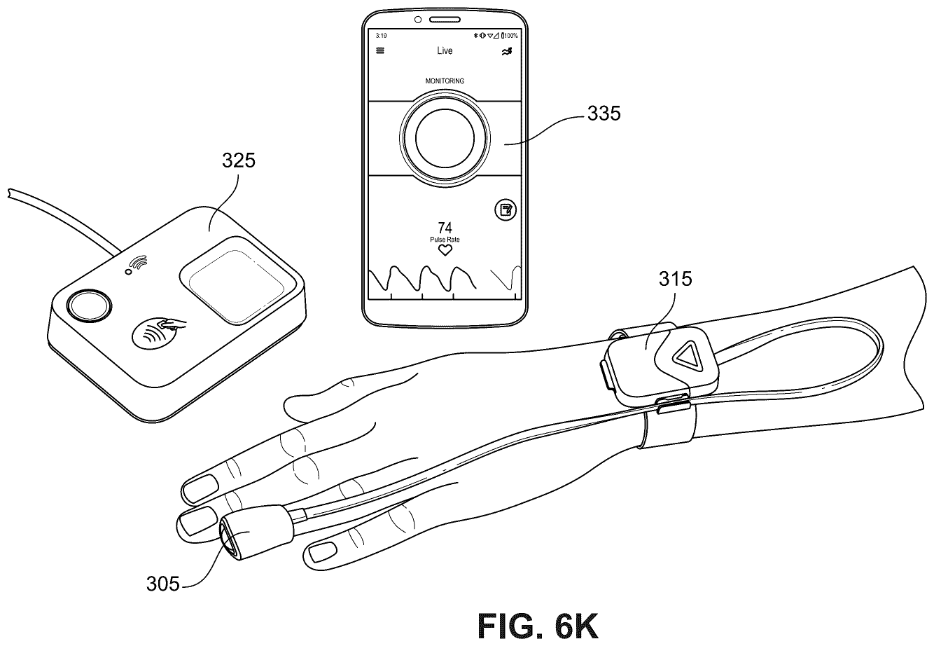

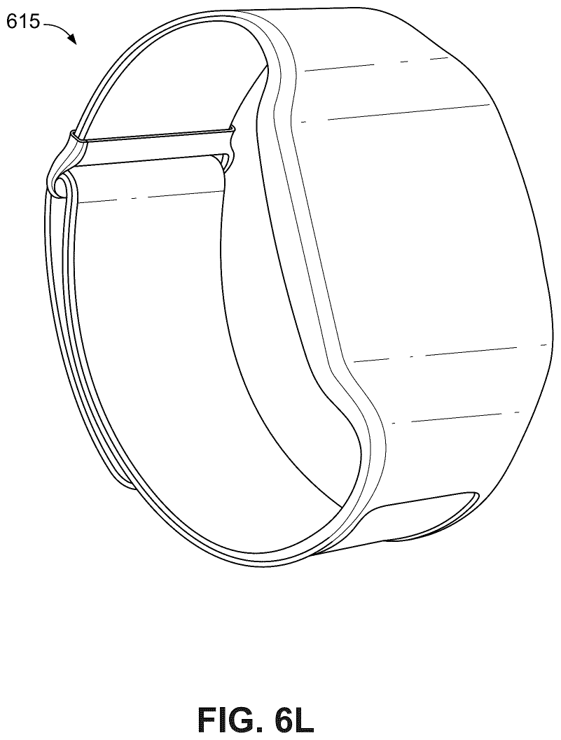

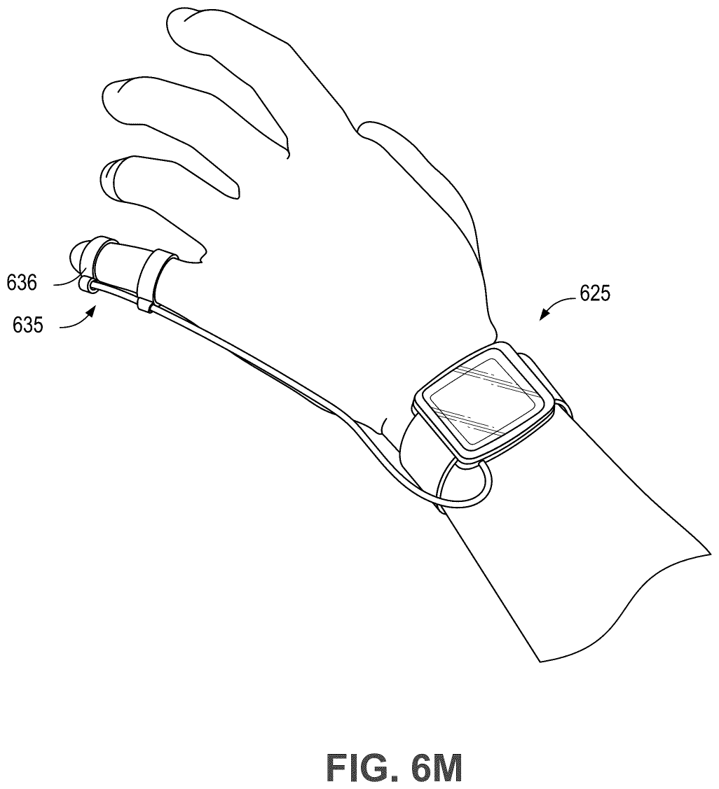

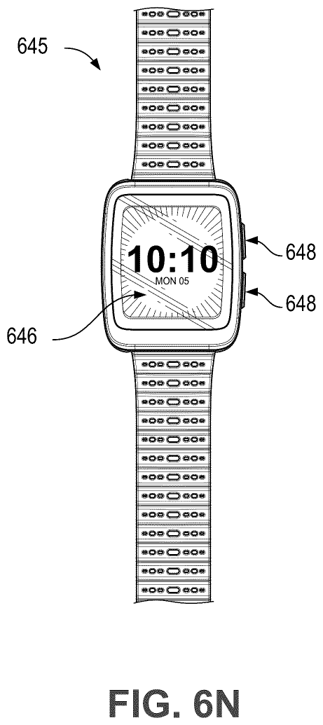

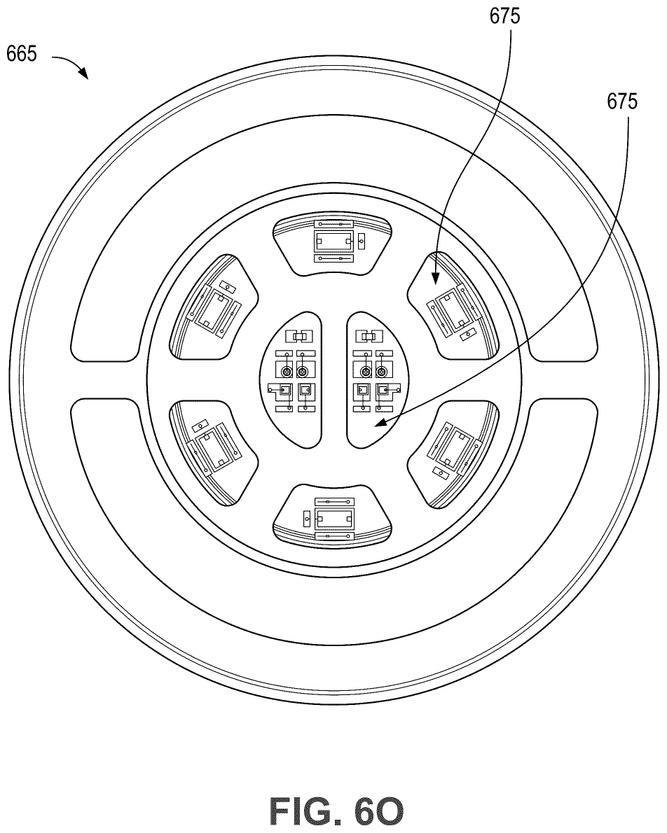

FIG. 1C is an overview of another example opioid use monitoring system. As illustrated above in FIG. 1A, the opioid users' support network can include friends, family, emergency services, care providers, and overdose care networks, for example, that communicate over a network, such as the Internet. The support network receives notifications and/or status updates of the opioid user's condition. A monitoring device including a sensor can monitor the opioid user's respiration and other biological parameters, such as heart rate, blood oxygen saturation, perfusion index, for example, and provide the parameters to a HUB device that can communicate over the network. An example of a HUB device is illustrated in FIG. 6H. The HUB device receives the sensor data from the sensor. The HUB device can send the sensor data over the network to the server. The HUB device can at least partially process the sensor data and sends that at least partially processed sensor data to the server. The server processes the sensor data or the at least partially processed sensor data and determines whether an overdose event is imminent and/or occurring. When an overdose event is imminent and/or occurring, the server notifies the support network and the mobile application on the opioid user's mobile device.

### Instrumentation-Sensor and Signal Processing Device

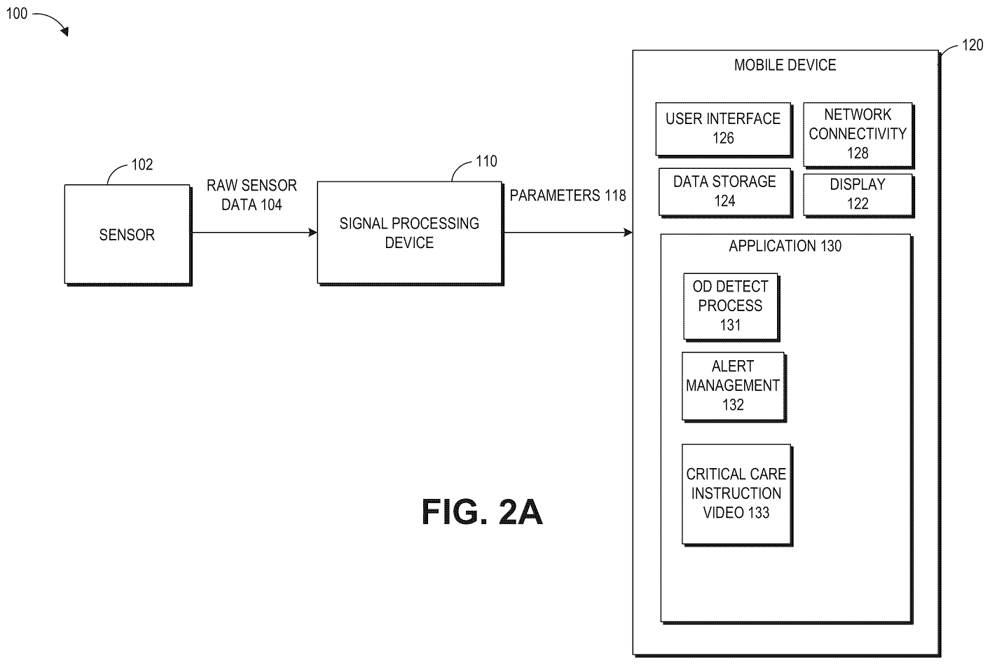

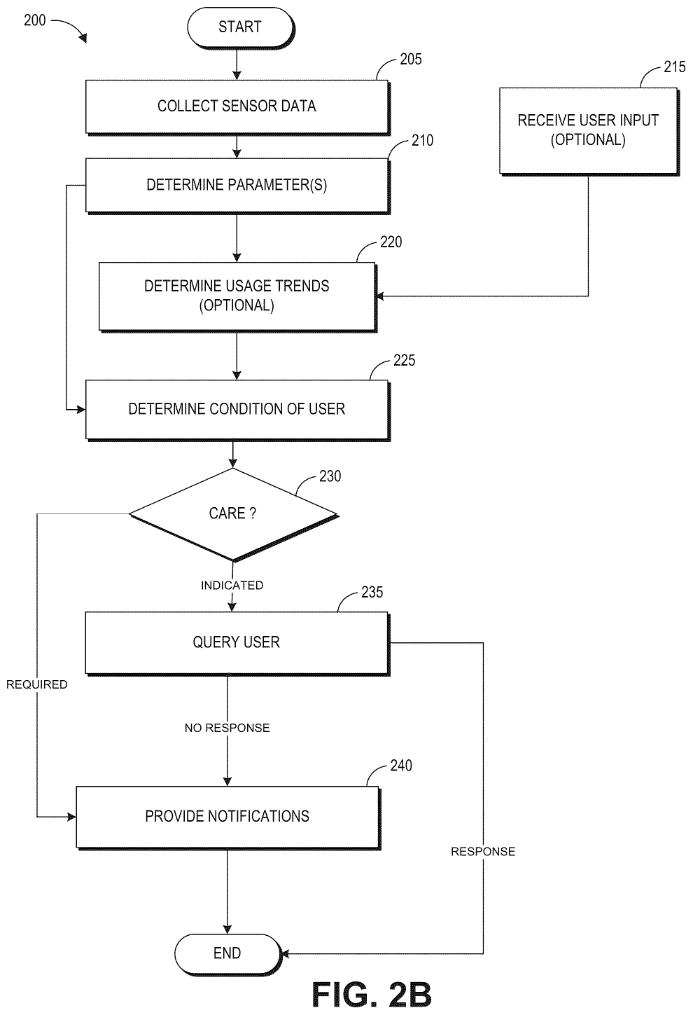

FIG. 2A illustrates an example physiological monitoring system 100. The illustrated physiological monitoring system 100 includes a sensor 102, a signal processing device 110, and a mobile computing device 120.

The sensor 102 and the signal processing device 110 can comprise a pulse oximeter. Pulse oximetry is a noninvasive method for monitoring a person's oxygen saturation. The sensor 102 is placed on the user's body and passes two wavelengths of light through the body part to a photodetector. The sensor 102 can provide raw data 104 to the signal processing device 110, which determines the absorbance's of the light due to pulsating arterial blood. The pulse oximeter generates a blood-volume plethysmograph waveform from which oxygen saturation of arterial blood, pulse rate, and perfusion index, among other physiological parameters, can be determined, and provides physiological parameters 118 to the mobile computing device 120.

The pulse oximeter can be transmissive, where the sensor 102 is placed across a thin part of the user's body, such as a fingertip or carlobe, for example, or reflective, where the sensor 102 can be placed on the user's forehead, foot, or chest, for example.

The sensor 102 and the signal processing device 110 can be packaged together. The sensor 102 can be not packaged with the signal processing device 110 and communicates wirelessly or via a cable with the signal processing device 110.

Examples of pulse oximeters are the MIGHTYSAT RX fingertip pulse Oximeter®, the Rad-57® handheld pulse CO-oximeter, and the Rainbow® CO-oximeter, all by Masimo Corporation, Irvine, CA, which are capable of being secured to a digit, such as a finger.

Because opioid users may want to be discrete when monitoring opioid use for indications of an overdose event, sensors 102 that are not visible may provide additional confidentiality for the user. The sensor 102 can be applied to a toe and the signal processing device 110 can comprise an ankle brace. The sensor 102 can be a ring on the user's finger or a bracelet on the user's wrist, and the signal processing device 110 can be within an arm band hidden under the user's sleeve. The sensor 102 or the sensor 102 and the signal processing device 110 can be integrated into a fitness device worn on the user's wrist. Such pulse oximeters can be reflective or transmissive. The sensor 102 can be an car sensor that is not readily visible.

Other varieties of sensors 102 can be used, for example adhesive sensors, combination reusable/disposable sensors, soft and/or flexible wrap sensors, infant or pediatric sensors, multisite sensors, or sensors shaped for measurement at a tissue site such as an car.

Other sensors 102 can be used to measure physiological parameters of the user. For example, a modulated physiological sensor can be a noninvasive device responsive to a physiological reaction of the user to an internal or external perturbation that propagates to a skin surface area. The modulated physiological sensor has a detector, such as an accelerometer, configured to generate a signal responsive to the physiological reaction. A modulator varies the coupling of the detector to the skin so as to at least intermittently maximize the detector signal. A sensor processor controls the modulator and receives an effectively amplified detector signal, which is processed to calculate a physiological parameter indicative of the physiological reaction. A modulated physiological sensor and corresponding sensor processor are described in U.S. Publication No. 2013/0046204 to Lamego et al., filed Feb. 21, 2013, titled “MODULATED PHYSIOLOGICAL SENSOR” and assigned to Masimo Corporation, Irvine, CA, which is hereby incorporated by reference herein.

The sensor 102 can include an electroencephalograph (“EEG”) that can be configured to measure electrical activity along the scalp. The sensor 102 can include a capnometer or capnograph that can be configured to measure components of expired breath.

An acoustic sensor 102 can be used to determine the user's respiration rate. An acoustic sensor utilizing a piezoelectric device attached to the neck is capable of detecting sound waves due to vibrations in the trachea due to the inflow and outflow of air between the lungs and the nose and mouth. The sensor outputs a modulated sound wave envelope that can be demodulated so as to derive respiration rate. An acoustic respiration rate sensor and corresponding sensor processor is described in U.S. Publication No. 2011/0125060 to Telfort et al., filed Oct. 14, 2010, titled “ACOUSTIC RESPIRATORY MONITORING SYSTEMS AND METHODS” and assigned to Masimo Corporation, Irvine, CA, which is hereby incorporated by reference herein.

The mobile computing device 120 can include an accelerometer that is configured to detect motion of the mobile computing device 120. When the user holds the mobile computing device 120 or attaches the mobile computing device 120 to his clothing in such a way that the accelerometer detects motion of the user, then the accelerometer can be used to detect lack of motion of the user. The lack of user motion can be used to determine the user's condition, as described below.

When the user holds the mobile computing device 120, the accelerometer can sense vibrations from the user indicative of the user's heart rate. A lack of vibrations sensed by the accelerometer can indicate no heart rate and reduced occurrences of vibrations sensed by the accelerometer can indicate cardiac distress. The indications of cardiac activity sensed by the accelerometer in the mobile computing device can be used to determine the user's condition, as described below.

The sensor 102 can be a centroid patch worn by the user that includes an accelerometer. Data indicative of the movement of the accelerometer can be transmitted wirelessly to the mobile computing device 120. Based on movement detected by the accelerometer, the application detects the respiration rate of the user. An oxygen sensor configured to monitor the user's breath can wirelessly transmit an indication of the oxygen present in the user's exhaled breath.

The physiological sensor 102 and the mobile computing device 120 can be connected via a cable or cables and the signal processing device 110 can be connected between the sensor 102 and the mobile computing device 120 to conduct signal processing of the raw data 104 before the physiological parameters 118 are transmitted to the mobile computing device 120. A mobile physiological parameter monitoring system is described in U.S. Pat. No. 9,887,650 to Muhsin et al., issued on Jan. 30, 2018, titled “PHYSIOLOGICAL MONITOR WITH MOBILE COMPUTING DEVICE CONNECTIVITY”, and assigned to Masimo Corporation, Irvine, CA, which is hereby incorporated by reference herein.

In various oximeter examples, the sensor 102 provides data 104 in the form of an output signal indicative of an amount of attenuation of predetermined wavelengths (ranges of wavelengths) of light by body tissues, such as, for example, a digit, portions of the nose or car, a foot, or the like. The predetermined wavelengths often correspond to specific physiological parameter data desired, including for example, blood oxygen information such as oxygen content (SpOC), oxygen saturation (SpO₂), blood glucose, total hemoglobin (SpHb), methemoglobin (MetHb), carboxyhemoglobin (SpCO), bulk tissue property measurements, water content, pH, blood pressure, respiration related information, cardiac information, perfusion index (PI), pleth variability indices (PVI), or the like, which can be used by the mobile computing device 120 to determine the condition of the user. Sensor data 104 can provide information regarding physiological parameters 118 such as EEG, ECG, heart beats per minute, acoustic respiration rate (RRa), breaths per minute, end-tidal carbon dioxide (EtCO₂), respiratory effort index, return of spontaneous circulation (ROSC), or the like, which can be used to determine the physiological condition of the user.

Referring to FIG. 2A, the sensor 102 can transmit raw sensor data 104 to the signal processing device 110, and the signal processing device 110 can convert the raw sensor data 104 into data representing physiological parameters 118 for transmission to the mobile computing device 120 for display, monitoring, and storage. The sensor data 104 can be transmitted wirelessly, using Bluetooth®, near field communication protocols, Wi-Fi, and the like or the sensor data 104 can be transmitted to the signal processing device 110 through a cable.

The sensor data 104 can be corrupted by noise due to patient movement, electromagnetic interference, or ambient light, for example. The physiological parameter monitoring system 100 can apply noise filtering and signal processing to provide the physiological parameters 118 for analysis and display on the mobile computing device 120. Such complex processing techniques can exceed the processing capabilities of the mobile computing device 120, and therefore the signal processing device 110 can handle signal processing of the raw sensor data 104 and transmit the processed physiological parameters 118 to the mobile computing device 120.

In the context of pulse oximetry, the signal processing device 110 can use adaptive filter technology to separate an arterial signal, detected by a pulse oximeter sensor 102, from the non-arterial noise (e.g., venous blood movement during motion). During routine patient motions (shivering, waving, tapping, etc.), the resulting noise can be quite substantial and can easily overwhelm a conventional ratio based oximetry system. This can provide accurate blood oxygenation measurements even during patient motion, low perfusion, intense ambient light, and electrocautery interference. Accordingly, false alarms can be substantially eliminated without sacrificing true alarms.

The signal processing device 110 can transmit the physiological parameters 118 wirelessly, using Bluetooth®, near field communication protocols, Wi-Fi, and the like to the mobile computing device 120, or the signal processing device 110 can transmit the physiological parameters 118 to the mobile computing device 120 through a cable.

FIGS. 6A-6J illustrate various example sensors 102 and signal processing devices 110. FIG. 6A illustrates a mobile physiological monitoring system 610 that includes a fingertip pulse oximeter sensor 102 that is connected to the mobile computing device 120, which is illustrated as a smartphone, through a cable that includes the signal processing device 110.

FIGS. 6B-6D illustrate other example mobile physiological sensor assemblies that can be in physical communication with a user to collect the user's physiological data and send indications of the user's physiological parameters to the mobile computing device 120. FIG. 6B illustrates a mobile physiological sensor assembly 620 that includes an electroencephalograph (“EEG”) that can be configured to measure electrical activity along the scalp. FIG. 6C illustrates a mobile physiological sensor assembly 630 that includes a capnometer or capnograph that can be configured to measure components of expired breath. FIG. 6D illustrates a mobile physiological sensor assembly 640 that includes an acoustic respiratory monitor sensor that can be configured to measure respiration rate using an adhesive sensor with an integrated acoustic transducer.

FIG. 6E illustrates the Rad-57® handheld pulse CO-oximeter 650 by Masimo Corporation, Irvine CA. The oximeter 650 has a fingertip oximeter sensor 102 that communicates the raw data 104 through a cable to the signal processing device 110, which includes display capabilities.

FIG. 6F illustrates the MIGHTYSAT RX fingertip pulse Oximeter® 660 by Masimo Corporation, Irvine, CA. The sensor 102 and the signal processing device 110 of the oximeter 660 are integrated into a single package.

FIG. 6G illustrates a physiological parameter assembly 670 comprising a sensor 102 applied to the toe and a signal processing device 110 in an ankle band for discreetly monitoring for opioid overdose conditions.

FIG. 6H illustrates a monitoring hub 680 comprising a ROOT® monitoring hub 326 with a Radical-7® pulse oximeter 200, both by Masimo Corporation, Irvine, CA. The medical monitoring hub 680 can expand monitoring capabilities by bringing together signal processing and display for multiple physiological parameters, such as brain function monitoring, regional oximetry, and capnography measurements.

FIG. 6I illustrates a physiological parameter assembly 690 comprising a sensor 102 and a signal processing device 110 that can be worn as a glove. When the glove is placed on the user's hand, the sensor 102 can be placed on one of the fingertips. The sensor 102 can be a disposable sensor. The sensor 102 can be built inside or outside the fingers of the glove. The sensor 102 can be integrated to the fingers of the glove. The cable of the signal processing device 110 can be integrated to the glove. Advantageously, the glove is easy to wear, stays in place, and can be easily removed when the user is not in need of opioid overdose monitoring. The glove 690 can fasten at the wrist with a strap, hook and loop fastener, and the like. The sensor 110 can be wireless and communicates with the mobile device 120 using wireless technology, such as Bluetooth®, and the like.

FIG. 6J illustrates a physiological parameter assembly 695 comprising a sensor 102 and a cable for connection to a signal processing device. The sensor 102 can be a disposable sensor. The sensor 102 can be placed around a finger. The sensor 102 can communicate sensor data wirelessly.

FIG. 6K illustrates an example physiological data monitoring system 685 that can utilize any of the risk score determination engines mentioned herein. The risk score determination engine can be designed to help family and friends identify the symptoms of an opioid overdose by detecting physiological markers present during opioid-induced respiratory depression and ideally, helping them know when it's time to intervene—for example, by administering a potentially life-saving dose of naloxone. The physiological monitoring system and risk score determination engine can be used at home or in the hospital or another care setting, by patients prescribed opioids after surgery or managing a chronic or prolonged condition, as well as people suffering from opioid use disorder.

In one implementation, the system 685 can include four components: 1) a sensor 305 able to attach to a body part such as a tetherless, adhesive fingertip sensor; 2) a reusable physiological monitor device 315 (e.g., a pulse oximeter) with wireless capabilities (e.g., wireless chip such as a Bluetooth chip); 3) a monitoring hub 325 having one or more processors; and 4) a smart device application 335. The sensor 305 can provide real-time monitoring for opioid-induced respiratory depression, enabled by the pattern recognition algorithm of the risk score determination engine and wireless capabilities-even during movement, when hands are cold, and on all skin pigmentations. Data from the sensor 305 and wireless capabilities are wirelessly relayed to the monitoring hub 325 and the smart device application 335, which continuously analyzes the user's physiological data for trends and patterns associated with the physiology of an opioid-induced respiratory depression event to quantify the risk of an opioid overdose. As the level of risk rises, the app and hub provide alerts. Upon early onset, an audible and visual alarm, designed to trigger early intervention opportunities for the user to self-recover or get help, is provided. If the risk score determination engine risk score continues to worsen, in addition to the repeated alarms, automatic texts are sent to designated friends and family members, letting them know it may be time to intervene, for example by administering naloxone or taking other action. Finally, if the severity of the risk level progresses even further, there is an optional setting that can be activated during setup that enables a service center to place an automatic wellness call to the user, the outcome of which may lead to EMS being dispatched.

FIG. 6L illustrates an example sleep band 615 for monitoring physiological data that can utilize any of the risk score determination engines mentioned herein. As mentioned above, the sleep band 615 can provide physiological data to the risk score determination engines. For instance, the sleep band 615 can continuously or intermittently provide physiological data. In some implementation, the sleep band 615 can comprise a reusable chip that snaps into a sensor which can also communicate with a remote monitoring hub.

Aspects disclosed herein provide an escalating alarm upon the detection of an opioid overdose event. FIGS. 6M-60 illustrate a wearable device, such as a watch, that can provide the escalating alarm. The wearable device can include a sensor to sense physiological parameters of the wearer. The alarm can escalate. For example, initially the alarm can provide the wearer with an indication that an opioid overdose event is occurring or will soon occur. Should no action be taken by the wearer of the wearable device, the alarm can escalate to attract the attention of bystanders. The wearable device can provide audible instructions for the bystander to follow. The instructions can include instructions to wake or shake the wearer, instructions to administer an opioid receptor antagonist, such as Naloxone, to reverse the effects of the opioid overdose. The alarm can escalate to wake the wearer. In an aspect, the wearable device may communicate with another wearable device to case the second wearable device to alarm to notify friends and family of the opioid overdose event.

FIG. 6M illustrates an example fingertip sensor that can be coupled to a wearable device 625. FIG. 6M illustrates a non-limiting example of the second sensor 635 that is a fingertip sensor. The second sensor 635 can comprise a blood pressure sensor 636. The second sensor 635, in some implementations, can comprise sensor 305 shown in FIG. 6K. The second sensor 635 can extend from the wearable 625 device as shown in FIG. 6M or any of the wearable device examples disclosed herein.

FIG. 6N shows an example wearable device 645 including a display 646 and buttons 648. The display 646 may be configured to display many different screens. For example, in some embodiments, the display 646 may display a screen with various physiological parameter information (such as values and trends) and in other embodiments, the display 646 may display a screen with no physiological parameter information. In some embodiments, the display 646 may display a screen with non-physiological related information such as date, time, and other notifications. In another example, the display 646 can display the alarm indication, as well as the wearable device 645 providing the escalating audible alarm. The wearer can also be informed of physiological parameters, such as vital signs including but not limited to pulse rate (may be measured from two different sources including ECG and PPG), and oxygen saturation by the wearable device 645. The wearable device 645 can display one or more of the measured physiological parameters on its display 646. The information can be helpful in providing feedback to the wearer and/or a third party user, for example, a healthcare professional or the wearer's family member, when the wearer is exercising, or otherwise for warning the wearer of possible health-related conditions, including but not limited to changes in the wearer's physiological parameters in response to medication that is being administered to the wearer.

FIG. 6O illustrates a top view of an example embodiment of a physiological parameter measurement sensor or module 665 incorporated into the wearable device such as wearable device 645 shown in FIG. 6N. The wearable device 645 can include the physiological parameter measurement sensor or module 665 configured to measure an indication of the wearer's physiological parameters, which can include, for example, pulse rate (PR), respiration rate, oxygen saturation (SpO₂), Pleth Variability Index (PVI), Perfusion Index (PI), Respiration from the pleth (RRp), hydration, glucose, blood pressure, resting PR, desaturation, acoustic data (e.g., microphone and/or phonocardiography) and/or other parameters. The physiological parameter measurement sensor or module 665 can be an optical sensor. Additionally, the module 665 can optionally calculate a wellness index based on more than one individual physiological parameter measured by the module 665 based on externally connected sensors and/or patient monitoring devices. The module 665 can perform intermittent and/or continuous monitoring of the measured parameters. The module 665 can additionally and/or alternatively perform a spot check of the measured parameters, for example, upon request by the wearer.

### Instrumentation-Mobile Computing Device

Any mobile computing device 120 that is compatible with the physiological parameter assembly that includes the sensor 102 and the signal processing device 110 can be used. A compatible mobile computing device can be one of a wide range of mobile devices such as, but not limited to a mobile communications device (such as a smartphone), laptop, tablet computer, netbook, PDA, media player, mobile game console, wristwatch, wearable computing device, or other microprocessor based device configured to interface with the signal processing device 110 and provide notifications based at least in part on the monitored physiological parameters 118.

Referring to FIG. 2A, the mobile computing device 120 can include a display 122 for display of the physiological parameters, for example in a user interface and/or software application, as discussed in more detail below. The display 122 can include a display screen such as an LED or LCD screen, and can include touch sensitive technologies in combination with the display screen. Mobile computing device 120 can include software configured to display some or all of the output measurement data on the display screen. The data display can include numerical or graphical representations of blood oxygen saturation, heart rate, respiration rate, pleth variability, perfusion index, and/or a respiratory efforts index, and may simultaneously display numerical and graphical data representations.

The mobile computing device 120 can include a user interface 126 that can receive user input. The user interface 126 can include buttons, a key pad, the touch sensitive technologies of the display screen 122, and other user input mechanisms typically found on the various example mobile computing devices 120.

The mobile computing device 120 can also include data storage 124, which can be configured for storage of the physiological parameters 118 and parameter history data and/or software applications that monitor the physiological parameters for an overdose indication and provide notifications. The storage 124 can be physical storage of the mobile computing device 120, and the storage 124 can be remote storage, such as on a server or servers of a data hosting service.

The mobile computing device 120 can also include a network connectivity feature 128 that provides network connection capabilities such as one or more of a cellular network, satellite network, Bluetooth, ZigBee, wireless network connection such as Wi-Fi or the like, and a wired network connection. The mobile computing device 120 can also include a data transfer port.

### Application Functionality Overview

The mobile computing device 120 can include software such as an application 130 configured to manage the physiological parameters 118 from the physiological parameter monitoring device 110. The application functionality can include trend analysis, current measurement information, alarms associated with above/below threshold readings, reminders to take measurement data at certain times or cycles, display customization, iconic data such as hearts beating, color coordination, bar graphs, gas bars, charts, graphs, or the like, all usable by a caregiver or application user to provide medical monitoring of specified physiological parameters. The display 122 can display the physiological parameters 118 as numerical values, graphs, charts, dials, and the like.

The application 130 via the mobile computing device 120 can also alert the user and/or person(s) designated by the user to an abnormal data reading. For example, an abnormally low blood oxygen saturation reading can cause the mobile computing device 120 to buzz, vibrate or otherwise notify the user of an abnormal reading, and to transmit a notification or alert to the user, the designated person(s) or medical personnel to a network via the network connectivity 128.

In addition, the application 130 includes one or more processes to monitor the physiological parameters 118 for the condition of the user, and in particular for signs of an opioid overdose. The application 130 can be set up by the user or a caregiver to notify another of the overdose event. This increases the likelihood that the opioid user, their immediate personal networks, and first responders are able to identify and react to an overdose by administrating medication used to reverse the effects of an opioid overdose, such as naloxone. Naloxone is an overdose-reversal drug. In some states, people who are or who know someone at risk for opioid overdose can go to a pharmacy or community-based program to get trained on naloxone administration and receive naloxone by “standing order,” which means a patient-specific prescription is not required. When administered in time, naloxone can restore an overdose victim's breathing long enough for trained medical assistance to arrive. In some instances, other overdose reversal drugs can be used, such as buprenorphine, and combination of buprenorphine and naloxone, and the like.

The application 130 can include processes and information to monitor and provide care to opioid users, such as, but not limited to an overdose detection process 131 configured to determine the condition of the user and whether medical care is indicated based at least on the physiological parameters 118, an alert management process 132 configured to manage alerts to the user and others in the user's network based at least in part on condition of the user, and information for the care/treatment for opioid use, such as a critical care instruction video 133.

### Opioid Overdose Monitoring

FIG. 2B illustrates an example process 200 to monitor physiological parameters 118 for opioid use and provide notifications. At block 205, the sensor 102 collects the raw data 104 from the user. In the case of a pulse oximeter sensor, the sensor 102 passes light, such as red and infrared light through a body part to a photodetector. The raw data 104 from the sensor 102 provides respiration information due to the absorbance of the light in the pulsating arterial blood.

At block 210, the signal processing device 110 receives the raw data 104 from the sensor 102, processes the raw data 104 to provide one or more parameters 118 to the mobile computing device 120. In the case of pulse oximetry, the signal processing device 110 generates a blood-volume plethysmograph waveform from which at least the peripheral oxygen saturation of arterial blood (SpO₂), respiration, pulse rate, and perfusion index (PI) may be determined. Other physiological parameters that may be determined are, for example, oxygen content (SpOC), blood glucose, total hemoglobin (SpHb), methemoglobin (MetHb), carboxyhemoglobin (SpCO), bulk tissue property measurements, water content, pH, blood pressure, cardiac information, and pleth variability indices (PVI). Sensor data 104 can provide information regarding physiological parameters 118 such as, for example, EEG, ECG, heart beats per minute, acoustic respiration rate (RRa), breaths per minute, end-tidal carbon dioxide (EtCO₂), respiratory effort index, and return of spontaneous circulation (ROSC).

### User Input

At block 215, the application 130 via the mobile computing device 120 can query the user and receive user input. The mobile computing device 120 can present questions on the display 122 and the user can reply using the user interface 126. For example, the user can be asked for the information on the prescription label, the dosage and/or frequency of the opioid being consumed and any other drugs the user is consuming. The mobile computing device 120 can ask the user to input his weight, age, and other physical attributes that may be factors in the user's reaction to the opioid and dosages of the medication, such as naloxone and the like, used to reverse the effects of an overdose. The mobile computing device 120 can ask whether the user is OK or in need of assistance. A response from the user can indicate that the user is conscious and not overdosed. The application 130 can ask the user for a response when the analysis of the parameters 118 indicates an overdose event, and if a response is received, indicating the user is conscious and not overdosed, the application 130 can refine the threshold used to determine an overdose event. The mobile computing device 120 can confirm the users name and location.

### Trends

At block 220, the application 130 can develop trends in the user's opioid usage using the physiological parameters 118 from past monitoring stored in the storage 124 as well as user input relating to weight, age, dosage, frequency, and additional drugs being consumed. The trends can be based on the parameters 118 and the user input, if any is received.

For example, opioid users that are also marijuana users can develop a greater tolerance for opioids. Further, opioids initially cause the perfusion index to increase due to vasodilation, then to decrease due to vasoconstriction. The increase and decrease of the perfusion index creates a perfusion profile. A user with a greater tolerance to opioids can have a different perfusion profile than a user that does not use marijuana in conjunction with opioids.

The application 130 can use the user input, if available, and stored physiological parameters, such as the perfusion profile, for example, and current physiological parameters to develop trends in the user's opioid usage and/or tolerance for opioids that can more accurately anticipate an overdose event. The application 130 can use past occurrences of “near misses” to further refine the conditions that may foreshadow an overdose event. A “near miss” is an event that provided indications of an overdose, such as an indication of respiration below a threshold, but did not result in an overdose event. The opioid dosage associated with a near miss can provide an indication of the user's tolerance to opioids and can be used by the application 130 to refine the determination of an imminent or occurring opioid overdose event.

By using the history of the physiological parameters 118 including the near-misses, and the user input, if available, the application 130 can learn which combination of events and parameter values indicate an overdose event may be imminent. Because time is of the essence in administrating medication, such as naloxone and the like, to reverse or reduce the effects of an overdose to an overdose victim, it is desirable to err in over-reporting, but too many false-positives of opioid notifications may desensitize responders. It is important that the application 130 learn the specific triggers for a specific user to increase accuracy in determining an overdose event for the specific user. The application 130 can learn the conditions leading up to an overdose event and refine its algorithm in order to notify others when help is needed and to discriminate against false-positive events.

The user's tolerance, as well as the user's physical attributes, such as weight and age, can be used by the application 130 to refine the quantity of medication that reverses or reduces the effects of an overdose, such as naloxone and the like, that should be administered to revive the user in an overdose event. The application 130 can monitor doses of the medication and report the dosages to clinicians who can determine whether the dosage is too high or too low.

The process 200 uses one or more of the user input, current physiological parameters, stored physiological parameters, “near miss” events, overdose events, to refine the indications of an overdose event so as to be able to more accurately determine the occurrence of an overdose event without notifying others of an overdose event that turns out to be false. Because time is of the essence in responding to an overdose victim, the application 130 may err on the side of over notification, but can learn the triggers for the specific user to avoid “crying wolf”, which may result in others ignoring the notifications.

### Data Analysis

At block 225, the application 130 determines the condition of the user based on one or more of the physiological parameters, user input, and trends. For example, the application 130 can compare the physiological parameters 118 against a threshold to determine is an overdose event is occurring or will soon occur. For example, opioids depress the user's breathing. If the one or more of the oxygen saturation, breaths per minute, perfusion index and respiratory effort index indicate respiratory failure but being less that a threshold, the application may determine that an overdose event has occurred. The threshold can be a predetermined threshold that is adjusted as the application 130 learns the overdose triggers associated with the user. As the application 130 develops the trends, the application can refine the thresholds for one or more of the physiological parameters 118.

The application 130 can use the user's perfusion index to determine the likelihood of an overdose event. For example, opioids initially cause the perfusion index to increase due to vasodilation, then to decrease due to vasoconstriction. This can be an identifiable perfusion profile that anticipates an overdose event.

The application 130 can use one or more physiological parameters 118 to determine the condition of the user. The application 130 can use one or more of the perfusion index (PI), respiration, and peripheral oxygen saturation (SpO₂) to determine the condition of the user. For example, the application 130 can use, but is not limited to, each of the perfusion index (PI), respiration, and peripheral oxygen saturation (SpO₂) alone; a combination of the PI, respiration, and SpO₂ together; a combination of PI and respiration; a combination of PI and SpO₂; or a combination of respiration and SpO₂ to determine the condition of the user. The analysis of the physiological parameters 118 may show that the physiological parameters are within normal ranges and the user is not in need of assistance or the analysis may indicate that an overdose event is imminent, is occurring, or has occurred.

Other physiological parameters 118 can be analyzed individually or in other combinations can be analyzed to determine whether the physiological parameters 118 of the user are within normal ranges or whether an overdose event is imminent, is occurring, or has occurred.

The application 130 can query the user to determine the condition of the user. No response from the user can indicate that the user is unconscious and can trigger an overdose event notification or alarm. As indicated above, a response from the user can indicate that the user is conscious and the information can be used by the application 130 to refine the changes in the user's physiological parameters 118 that indicate an opioid overdose is occurring or will occur soon.

As described above, the mobile computing device 120 can include an accelerometer that can detect user motion. A lack of user motion sensed by the accelerometer can indicate that the user in unconscious and can trigger an overdose event notification or alarm. Motion sensed by the accelerometer can indicate that the user is conscious and the information can be used by the application 130 to refine the changes in the user's physiological parameters 118 that indicate an opioid overdose is occurring or will occur soon.

As described above, the mobile computing device 120 can include an accelerometer that can sense vibrations from the user indicative of the user's heart rate. A lack of vibrations sensed by the accelerometer can indicate no heart rate and reduced occurrences of vibrations sensed by the accelerometer can indicate cardiac distress, which can trigger an overdose event notification or alarm. Heart rate within normal parameters can indicate that the user is not in need of assistance due to an overdose event.

At block 230, the application 130 can determine whether care is useful based on the condition of the user. If care is indicated, such that the physiological parameters indicate depressed respiration, but not at a life-threatening level, the application moves to block 235. At block 235, the application 130 queries the user. If a response is received, the process 200 moves to the END block. A response indicates that the user is conscious and not in need if immediate aid.

If, at block 230, the application 130 determines that care is required because the evaluation of the physiological parameters 118 indicate a life-threatening condition, the process 200 moves to block 240. In addition, if no response is received from the user query at block 235, the process 200 moves to block 240.

### Notifications

At block 240, the application 130 provides notifications based at least in part of the condition of the user. For example, the application 130 can display on the display 122 the user's physiological parameters, such as one or more of oxygen saturation, heart beats per minute, breaths-per-minute, pleth variability, perfusion index, and respiratory effort. The physiological parameters 118 can be displayed as charts, graphs, bar charts, numerical values, and the like. The application 130 can display trends in the physiological parameters 118.

The application 130 can provide notifications to selected friends indicating that there are no overdose conditions. The “everything is OK” notifications can be sent periodically or upon request. The “everything is OK” notifications can be sent during known exposure times. For example, the “everything is OK” notifications can be sent every 30 minutes from 6:00 PM when the user typically returns from work, to 11:00 PM when the user typically goes to sleep.

The application 130 can also report “near misses” to the caregiver. As described above, a “near miss” is an event that provided indications of an overdose, such as an indication of respiration below a threshold, but did not result in an overdose event.

Once the application 130 has determined that an overdose condition is imminent, is occurring, or has occurred, the application 130 can provide notification of the overdose to selected family, friends, caregivers, clinicians, and medical personnel. The notification can be sent to a crowd sourced community of users, friends, and medical personnel that look out for one another. The application 130 can provide the location of the user and/or directions to the user's location. The notification can include the location of the closest medical care and/or the location of the closest medication that reduces or reverses the effects of an overdose. Examples of such medications are, but not limited to, naloxone, buprenorphine, a combination of naloxone and buprenorphine, Narcan®, Suboxone®, Subutex®, and the like. The application 130 can indicate whether the overdose victim is conscious or unconscious.

The notification can include protocol for a first responder to render aid to the user. The application 130 can provide the user data to the medical personnel to aid them in administrating the correct dose of medication that reduces or reverses the effects of an overdose, such as naloxone and the like to the user. For example, if the overdose victim is also a heroin or marijuana user, the overdose victim may need a larger dosage of naloxone to reverse the effects of the opioid overdose than an overdose victim that does not also use heroin or marijuana. Further, the naloxone dosage may also need to be adjusted for the weight and age of the overdose victim. For example, a greater dosage on naloxone may be needed to reverse the depressed respiration effects of opioid overdose for an adult than is needed for a small child.

The application can provide trend data to medical personnel or to designated caregivers on a continual basis or may provide the trend data with the overdose notification. The dosage of medication to reduce or reverse the effects of the overdose, such as naloxone and the like, can be adjusted based at least in part on the trend data.

The application 130 can notify the user and request an acknowledgement for the user. For example, the application 130 can provide a visual notification on the display 122, and then cause the mobile computing device 120 to provide an audible notification, such as an audible alarm which can escalate to an increasing louder piercing sound in an attempt to wake up the user. The audible notification can include the name of the user. The application 130 can interact with a home system, such as Alexa®, Amazon Echo®, and the like, to create the alarm. The application 130 can cause the mobile computing device 120 or the home system, for example, to contact a live person who can provide immediate care instructions to the first responder.

The application 130 can provide the notifications to others in the user's community that have downloaded the application 130 on their mobile computing device. The application 130 can cause the mobile computing device 120 to send, for example, but not limited to text messages, emails, and phone calls to selected contacts in the user's mobile device 120, who may or may not have downloaded the application 130 to their mobile computing device 120. The mobile computing device 120 can automatically dial 911 or other emergency response numbers. The application 130 can transmit the location of the user to one or more selected ambulances and paramedics.

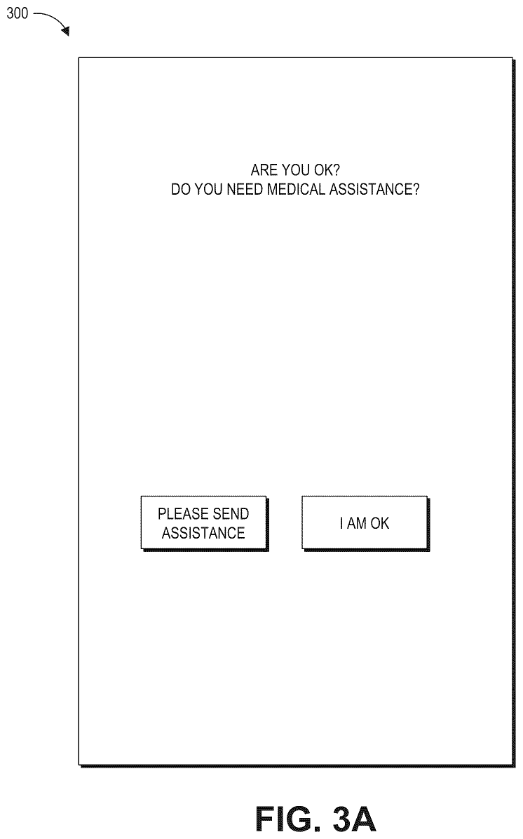

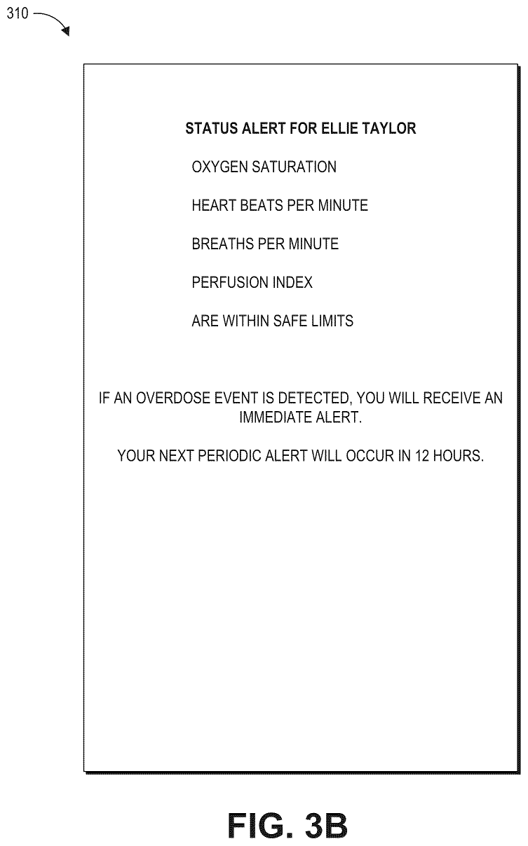

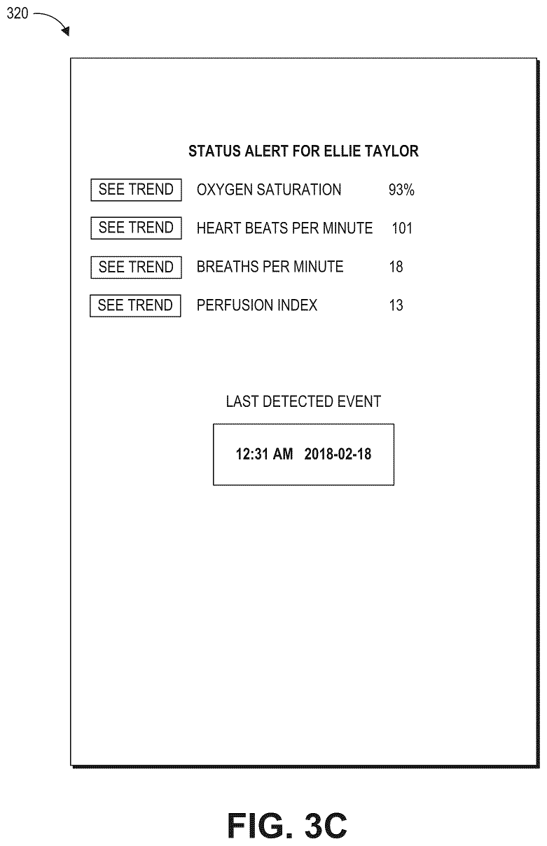

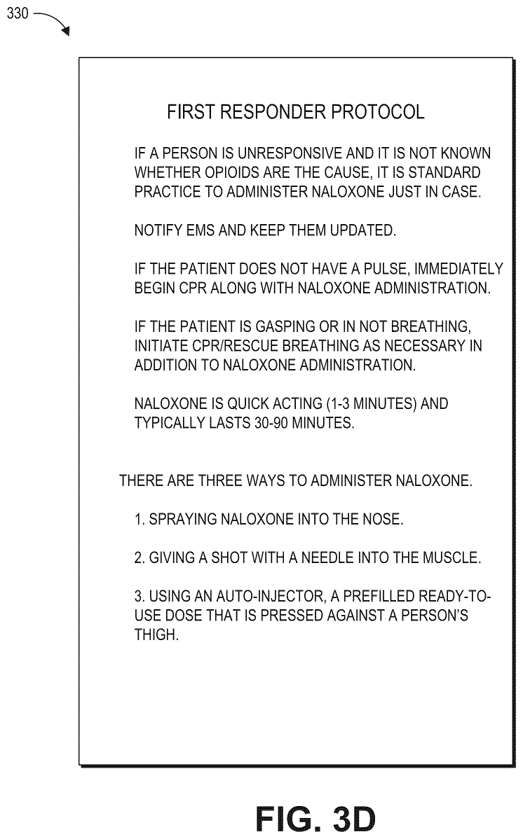

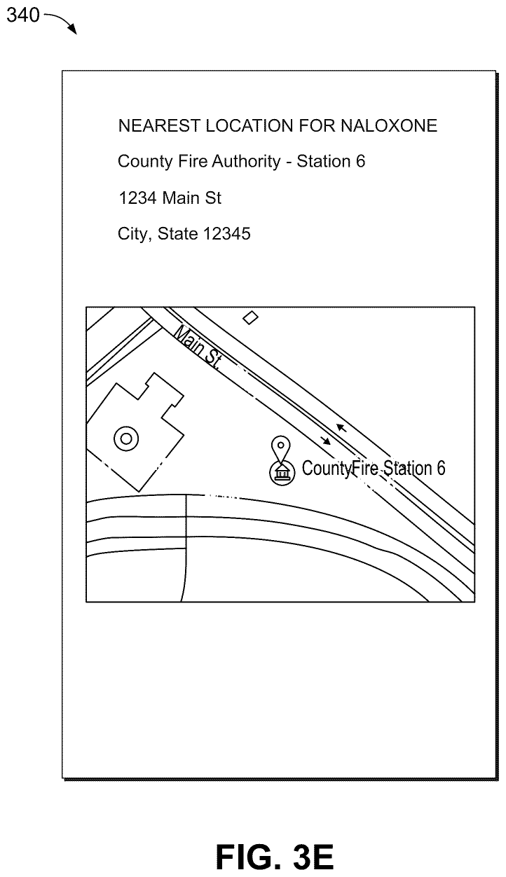

FIGS. 3A-3E illustrate various example software applications to provide information, notifications, and alerts to opioid users, first responders, medical personnel, and friends.

FIG. 3A is a screenshot 300 illustrating a request for user input. The illustrated screenshot 300 displays a question “ARE YOU OK? DO YOU NEED MEDICAL ASSISTANCE?” and selections for the user's response. If no response is received, the user may be assumed to be unconscious. If a response is received, the application 130 can use the physiological parameters 118 associated with the response to refine the algorithm to determine an overdose event for the specific user. The refinements can include refinements to the overdose threshold for the physiological parameters 118 or can include refinements to the parameter trends associated with an overdose event.

FIG. 3B is a screenshot 310 illustrating a periodic status alert that can be send via text message or email to friends or family that have set up periodic well checks for the user in the user's application 130. The illustrated screenshot 310 also indicates when the next well check will occur.

FIG. 3C is a screenshot 320 illustrating a status alert that can be send via text message or email to friends or family that have set up periodic well checks for the user in the user's application 130. The illustrated screenshot 320 indicates current values for monitored physiological parameters and provides a section SEE TRENDS to view the trend data for the physiological parameters. The illustrated screenshot 320 also indicates the date and time of the most recent overdose event.

FIG. 3D is a screenshot 330 illustrating first responder protocols. The illustrated screenshot 330 displays resuscitation information for the person(s) responding to the overdose notification.

FIG. 3E a screenshot 340 illustrating the nearest location to the user that has available naloxone. The illustrated screenshot 340 displays an address and a map of the location.

### Notify a Friend

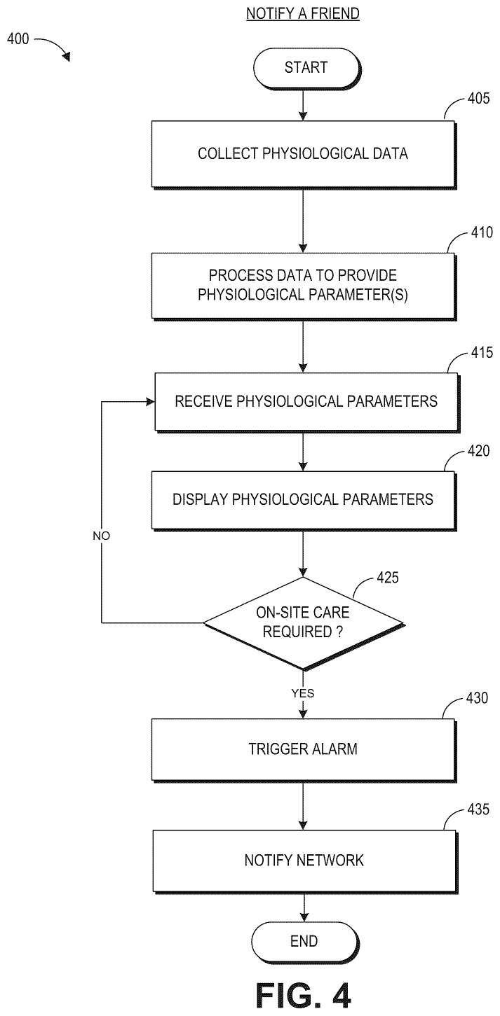

FIG. 4 illustrates an example process 400 to monitor for opioid overdose using the mobile physiological parameter monitoring system 100 including the sensor 102 and the signal processing device 110, and the mobile computing device 120. The user or the caregiver downloads the application 130 into the mobile computing device 120. The user or caregiver can select a person or persons to be notified by the mobile computing device 120 when the application 130 determines an opioid overdose event is occurring. The mobile computing device 120 can comprise a mobile communication device, such as a smartphone. The user attaches the sensor 102 to a body part, such as clipping the sensor 102 onto a finger, a toe, the forehead, for example, and connects either wirelessly or via a cable to the mobile computing device 120 that includes the application 130.

At block 405, the mobile physiological parameter monitoring system 100 collects raw data 104 from the sensor 102. At block 410, signal processing device 110 processes the raw data and provides the mobile computing device 120 with physiological parameters 118.

At block 415, the mobile computing device 120 receives the physiological parameters 118 from the physiological parameter monitoring device 110.

At block 420, the application 130 displays on the display 122 of the mobile computing device 120 the physiological parameters 118. The mobile computing device 120 can display numerical indications, graphs, pie charts, dials, and the like. The displays can include acceptable and unacceptable ranges for the physiological parameters 118. The display can be color coded. For example, acceptable ranges can be colored green and unacceptable ranges can be colored red. The application 130 can display on the mobile computing device 120 the physiological parameters 118 as the physiological parameters 118 are received (in real time) or at approximately the same time (near real time) as the physiological parameters 118 are received.

At block 425, the application 130 can monitor the physiological parameters 118 for indications of an opioid overdose. The monitored physiological parameters 118 can include the physiological parameters that are most likely affected by an overdose condition. The physiological parameters 118 can be one or more of the oxygen saturation, heart rate, respiration rate, pleth variability, perfusion index, and the like of the user.

The application 130 can determine whether the physiological parameters 118 indicate that the user needs on-site care. A blood oxygen saturation level below a threshold can indicate an opioid overdose condition. For example, the application 130 can monitor the oxygen saturation of the user and trigger an alarm when the oxygen saturation falls below a threshold. The application 130 can compare the user's current oxygen saturation level with a threshold that can indicate a minimum acceptable blood oxygen saturation level. An oxygen saturation level below the minimum acceptable blood oxygen saturation level can be an indication of an overdose event. For example, an oxygen saturation level below approximately 88 can indicate respiratory distress.

The application 130 can compare each of the monitored physiological parameters 118 with a threshold that indicates a minimum or maximum acceptable level for the physiological parameter 118. For example, the application 130 can compare the user's heart rate in beats per minute with the acceptable range of approximately 50 beats per minute to approximately 195 beats per minute. The application 130 can compare the user's respiration rate in breaths per minute with the acceptable range of approximately 6 breaths per minute to approximately 30 breaths per minute. The application 130 can compare the user's pleth the acceptable range of approximately 5 to approximately 40 and the user's perfusion index to a minimum acceptable perfusion index of approximately 0.3.

One or more physiological parameters 118 can be weighted and when the combination of weighted parameters falls below a threshold, the application 130 can trigger the notification of an opioid overdose event. One or more physiological parameters 118 can be weighted based on trends in the user's physiological parameters during opioid use and when the combination of weighted parameters falls below a threshold, the application 130 can trigger the notification of an opioid overdose event.

When the measured physiological parameters 118 are within acceptable ranges, the process 400 can return to block 415 and the mobile computing device 120 can continue to receive the physiological parameters 118 from the sensor 102 via the physiological parameter monitoring device 110. The application 130 can compare one, more than one, or all of the measured physiological parameters 118 to determine an overdose event.

When an overdose is indicated as imminent or occurring, the process 400 moves to block 430. For example, when the user's blood oxygen saturation level is at or below the threshold, the application 130 triggers an alarm at block 430. When at least one of the monitored parameters 118 is below an acceptable threshold, the process 400 can trigger an alarm. The alarm can be an audible alarm that increases in loudness, frequency, or pitch. The alarm can be the user's name, a vibration, or a combination of audible sound, vibration, and name.

The mobile computing device 120 can vibrate, audibly alarm, display a warning, visibly flash, and the like to notify the user or someone at the same physical location as the mobile computing device 120 to the overdose event. The alarm can be an audible alarm that increases in loudness, frequency, or pitch. The alarm can be the user's name, a vibration, or a combination of audible sound, vibration, and name.

The mobile computing device 120 can display the location of and/or direction to naloxone or other medication to reverse or reduce the effects of an overdose closest to the user. The mobile computing device 120 can display the phone number of the person associated with the closest medication to reverse or reduce the effects of an overdose, such as naloxone. The mobile computing device 120 can display resuscitation instructions to the first responder. The mobile computing device 120 can request an acknowledgement from the first responder. The mobile computing device 120 can display the resuscitation instructions to the first responder, call medical personnel, and facilitate questions and answers between the first responder and the medical personnel.

If the user is alone, this may not be enough to avoid a life-threatening overdose condition. At block 435, the application 130 can send a notification to the user's network, such as the person(s), emergency personnel, friends, family, caregivers, doctors, hospitals selected to be notified. The notification can be sent in conjunction with the network connectivity 128 of the user's mobile computing device 120. The notification informs the selected person(s) of the user's opioid overdose. For example, the selected person(s) can receive a notification on their mobile computing device. The selected person(s) can be a friend, a group of friends, first responders, medical personnel, and the like. The mobile computing device 120 can automatically dial 911 or other emergency response numbers.

The notification can be sent to a crowd sourced community of opioid users that look out for one another, such as a community of individuals and/or organizations associated with one or more opioid users. The community functions to provide help to opioid users and can includes not only other opioid users, but friends, family, sponsors, first responders, medics, clinicians, and anyone with access to medication to reverse or reduce the effects of an overdose, such as naloxone.

The notification can be one or more of text message, an automatically dialed phone call, an email, or the like. The notification can include one or more of a graphical representation, a numerical value, or the like of the user's unacceptable or out-of-acceptable-range physiological parameter 118, the time of the overdose, the location of the user, directions to the location, and the phone number of the user's mobile computing device 120. The notification can also provide the location of and/or direction to medication to reverse or reduce the effects of an overdose, such as naloxone, closest to the user, as well as the phone number of the person associated with the closest medication to reverse or reduce the effects of an overdose, such as naloxone.

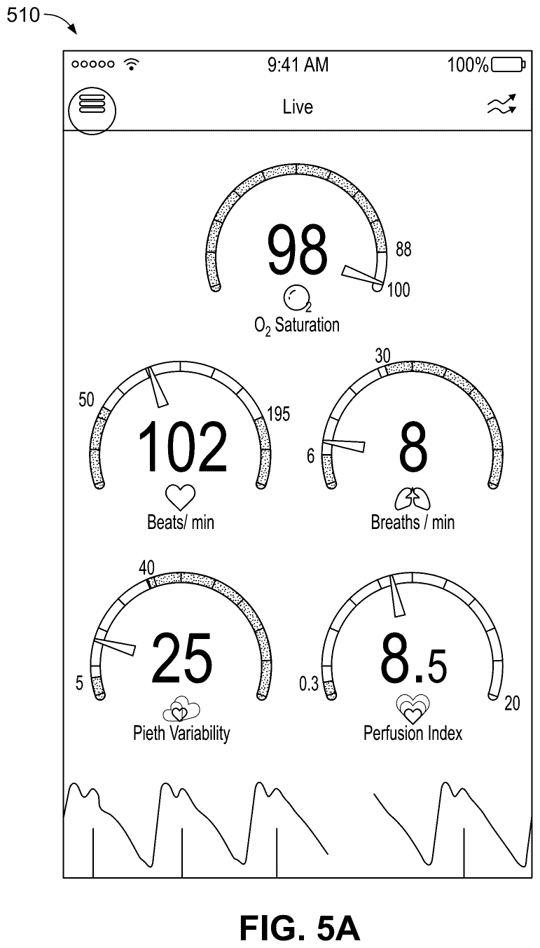

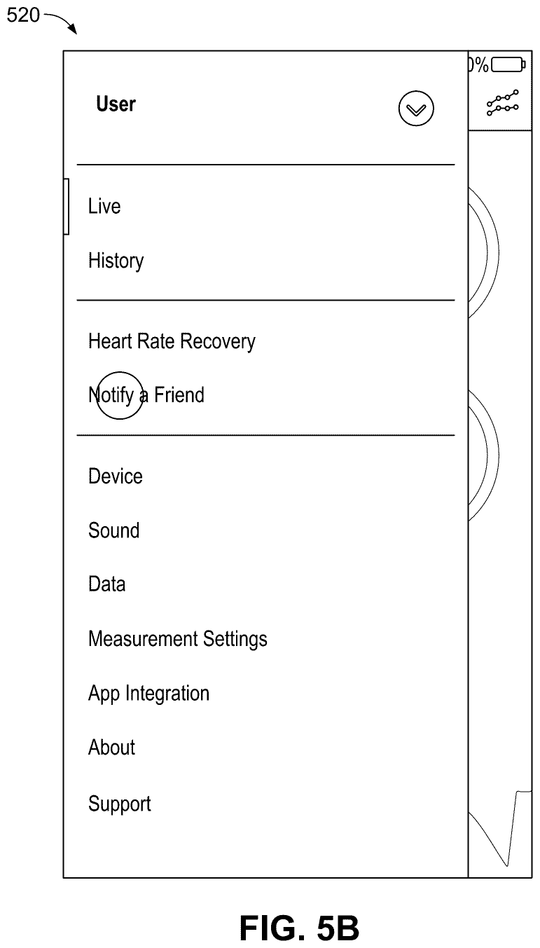

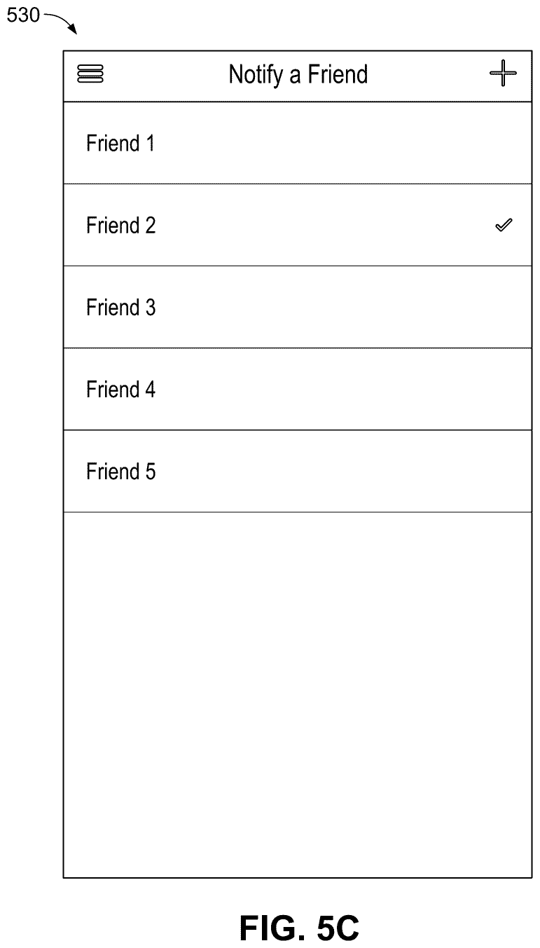

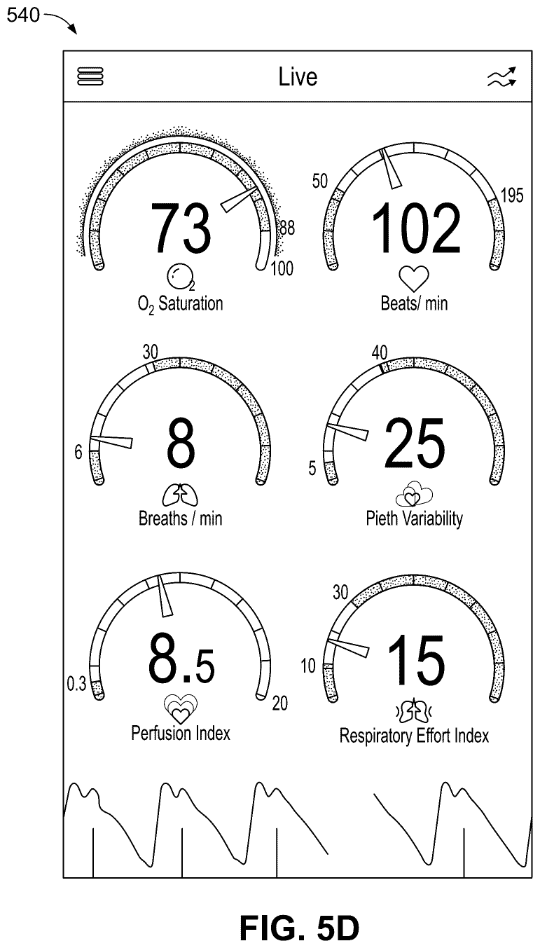

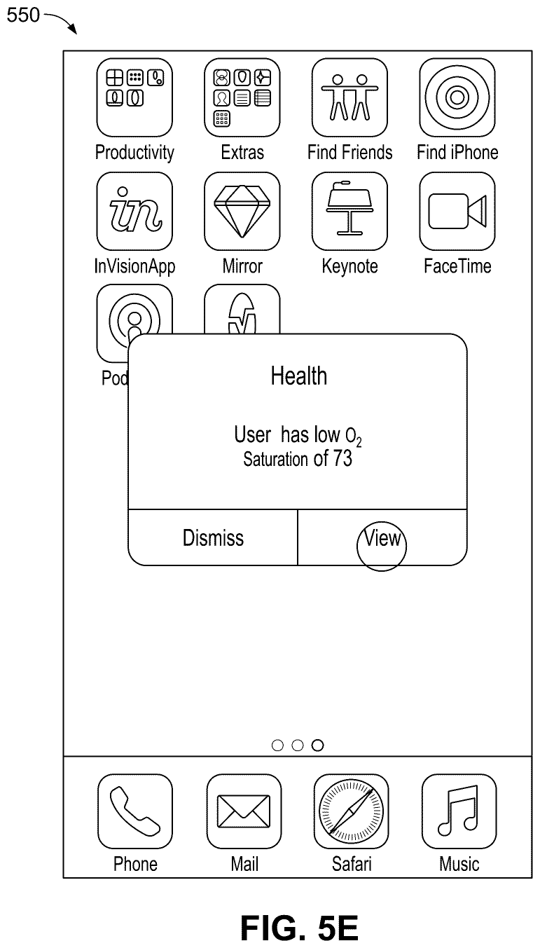

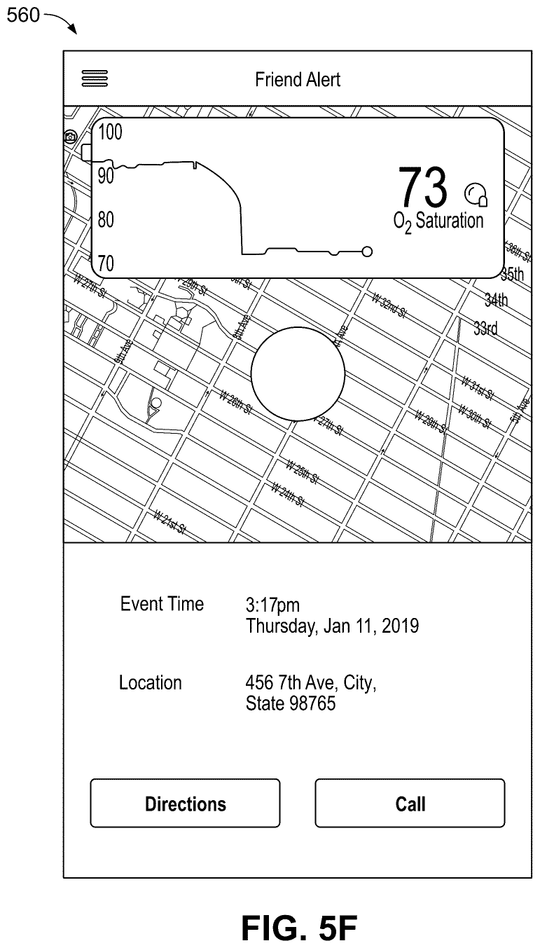

FIGS. 5A-5F illustrate various example software applications to trigger an alarm and notify a friend when an opioid overdoes is indicated.

FIG. 5A is an example screenshot 510 illustrating active monitoring of physiological parameters 118. The illustrated monitoring screenshot 510 displays the user's oxygen saturation, heart rate as beats per minute, respiration rate as breaths per minute, pleth variability and perfusion index. The physiological parameters 118 are represented as dials. The dials indicate a normal range and unacceptable ranges that can be above, below, or both above and below the normal range. A needle within the dial points to the current value of the physiological parameter and a numerical indication of the current value is displayed in the center of the dial.

FIG. 5B is an example screenshot 520 illustrating a home screen with the main menu. The illustrated home screen 520 includes a selection LIVE to display physiological parameters being monitored in real time or near real time, such as shown on the monitoring screenshot 510. The home screen 520 further includes a selection for HISTORY, HEART RATE RECOVERY, and NOTIFY A FRIEND.

Selecting HISTORY can display the past physiological parameters stored in storage 124 as one or more of graphs, charts, bar graphs, and the like. The application 130 can use the HISTORY to develop trends for the specific opioid user to more accurately determine when an opioid overdose event is imminent.

Heart rate is the speed of the heartbeat measured by the number of contractions of the heart per minute (bpm). The heart rate can vary according to the body's physical needs, including the need to absorb oxygen and excrete carbon dioxide. Selecting HEART RATE RECOVERY can display the recovery heart rate of the user after a near opioid overdose or overdose event.

Selecting NOTIFY A FRIEND allows the user or a caregiver to select a contact from the mobile computing device 120 to be notified in the event that the user's physiological parameters 118 indicate that the user is experiencing or will soon experience an overdose event.

The home screen 530 further includes a setup section that includes DEVICE, SOUND, DATA, MEASUREMENT SETTINGS, APP INTEGRATION, ABOUT, AND SUPPORT. The user can receive information, such as device data, for example, or select setting, such as what measurements are displayed, change alarm volume, and the like.

FIG. 5C is an example screenshot 530 illustrating the NOTIFY A FRIEND screen. The illustrated NOTIFY A FRIEND screen 530 allows the user or caregiver to select a person from the contacts stored on the mobile computing device 120 to be contacted when an overdose event occurs. In the illustrated NOTIFY A FRIEND screen 530, the second person on the contact list has been selected.

FIG. 5D is an example screenshot 540 illustrating live or active monitoring of the user having an alarm condition. The illustrated parameter monitoring screen 540 shows that the user's oxygen saturation level has dropped below an acceptable threshold of 88 to a value of 73. This indicates an overdose event may be occurring. The user's heart rate, respiration rate, pleth variability and perfusion index have not changed from the values displayed on the live monitoring screen 510.

FIG. 5D also includes a RESPIRATORY EFFORT INDEX, which provide an indication of whether breathing is occurring or is suppressed.

FIG. 5E is an example screenshot 550 illustrating a notification screen sent to the friend/selected contact to notify the friend of the user's overdose event. Once the alarm is triggered on the user's mobile computing device 120, the selected person is notified of the alarm status. The notification screen 550 can display the user's name and the alarm condition. The illustrated notification screen 550 informs the friend that Ellie Taylor has low oxygen saturation of 73. Selecting or touching the VIEW selection provides additional information.

FIG. 5F is an example screenshot 560 illustrating the friend alert including additional information provided to the selected person. The friend alert screen 560 can include the trend and current value of the alarming parameter. For example, the illustrated friend alert screen 560 displays the graph and current value of the user's oxygen saturation. The friend alert screen 560 can also display the user's location on a map, display the time of the initial alarm event, provide access to directions to the user from the friend's current location in one touch, and provide access to call the user in one touch. The friend has the knowledge that the user is overdosing and the information to provide help.

### Assistance for Responders and Caregivers

It is critical to administer an opioid receptor antagonist, such as Naloxone, to victims of opioid overdoses as soon as possible. Often it can be a matter of life or death for the overdose victim. As described herein, self-administrating delivery devices can administer the opioid receptor antagonist without user or responder action. Opioid overdose victims without a self-administrating delivery device rely on the responders, friends, or caregivers that are first on the scene to administer the opioid receptor antagonist. Assistance that can be provided to the first responders can be useful and the assistance can take many forms. The assistance can be visual or auditory indicators and/or instructions. The user can wear a band, such as a wrist band, for example, that changes color to indicate an opioid overdose event. A display, such as a display on a mobile device, can change color, or flash to draw attention when an opioid overdose event is detected. The mobile or other device can transmit a notification or transmit the flashing display to other devices within range to notify others of the opioid overdose event. The display can display instructions that explain how to administer the opioid receptor antagonist, such as Naloxone. The display can display instructions to wake the overdose victim using smelling salts, shaking, escalation of painful stimulation, loud noises, or any combination of these. The responder can be instructed to incrementally increase aggressive actions to wake the overdose victim. An example of incrementally increasing aggressive action can be loud sound, followed by a small amount of painful stimulation, followed by administration of a small amount of Naloxone or other opioid receptor antagonist, followed by an increased amount of painful stimulation. The first responder can be instructed to induce pain using acupuncture. The mobile or other device can speak the instructions to get the attention of others that are nearby. The mobile or other device can speak “Please inject Naloxone” to indicate urgency. The mobile or other device can beep to attract attention. The mobile or other device can buzz and/or provide voice directions to help in directionally finding the overdose victim.

The mobile or other device can provide codes to emergency personnel within proximity. The mobile or other device can send a signal to emergency personnel or police indicating that the Naloxone needs to be delivered as soon as possible.

The first responder can also administer medication to induce vomiting once the overdose victim is awake and upright. The user may regurgitate any opioid substances, such as pills, for example, that are still in the user's stomach.

### Network Environment

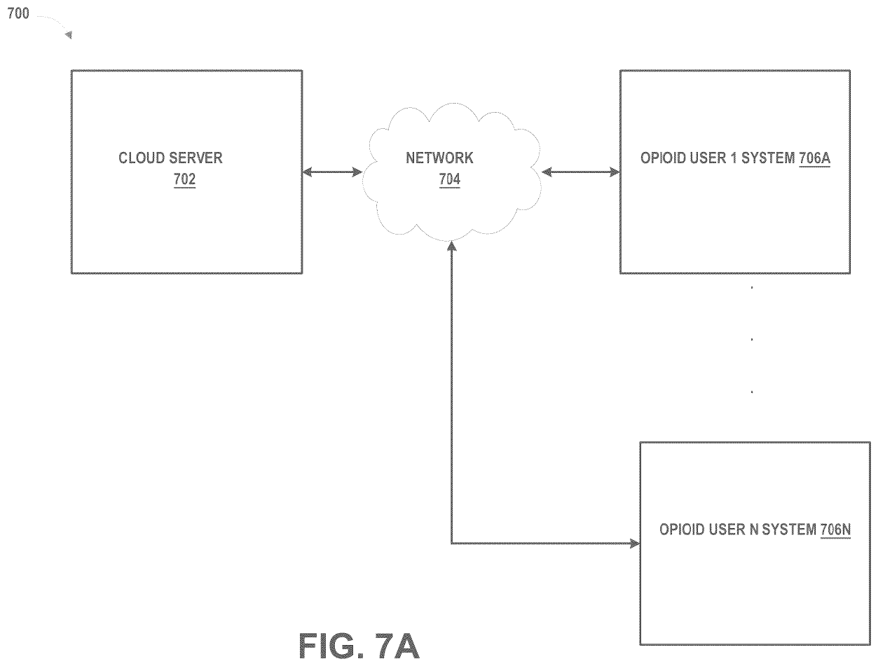

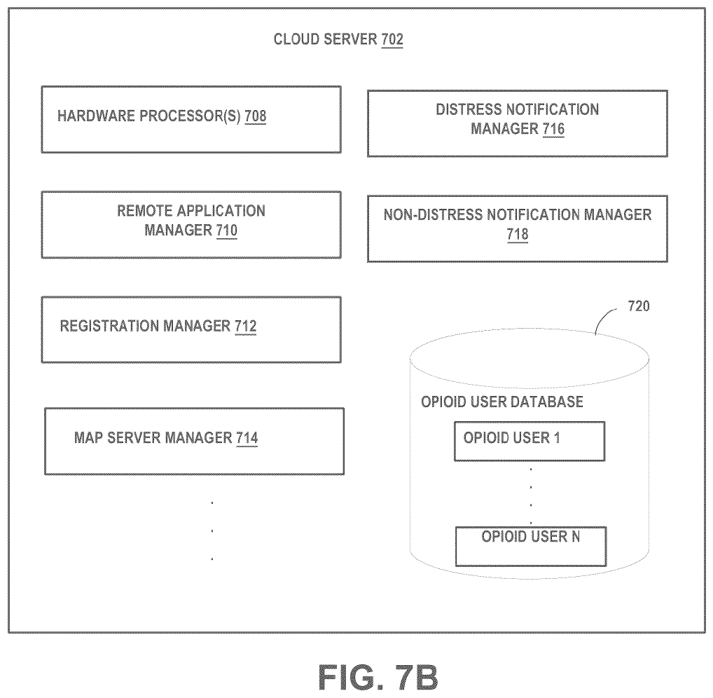

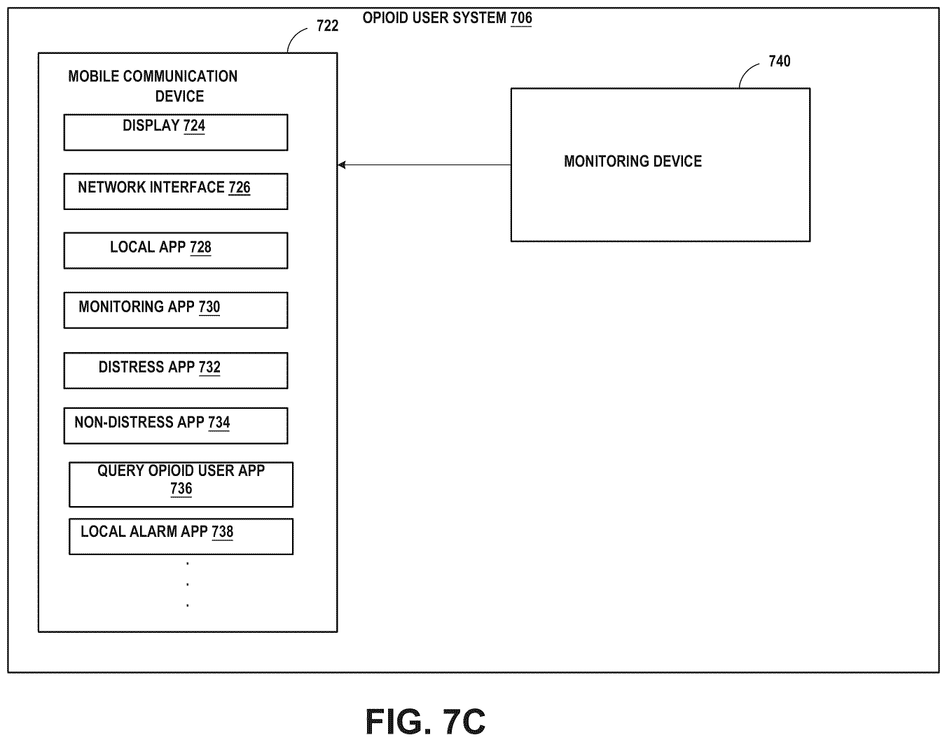

FIG. 7A illustrates an example network environment 700 in which a plurality of opioid user systems 706, shown as opioid user systems 706A . . . 706N, communicate with a cloud environment 702 via network 704. The components of the opioid user systems 706 are described in greater detail with respect to FIG. 7C.

The network 704 may be any wired network, wireless network, or combination thereof. In addition, the network 704 may be a personal area network, local area network, wide area network, over-the-air broadcast network (e.g., for radio or television), cable network, satellite network, cellular telephone network, or combination thereof. For example, the network 704 may be a publicly accessible network of linked networks such as the Internet. Protocols and components for communicating via the Internet or any of the other aforementioned types of communication networks are well known to those skilled in the art and, thus, are not described in more detail herein.

For example, the opioid user systems 706A . . . 706N and the cloud environment 702 may each be implemented on one or more wired and/or wireless private networks, and the network 704 may be a public network (e.g., the Internet) via which the opioid user systems 706A . . . 706N and the cloud environment 702 communicate with each other. The cloud environment 702 may be a cloud-based platform configured to communicate with multiple opioid user systems 706A . . . 706N. The cloud environment 702 may include a collection of services, which are delivered via the network 704 as web services. The components of the cloud environment 702 are described in greater detail below with reference to FIG. 7B.

FIG. 7B illustrates an example of an architecture of an illustrative server for opioid user monitoring. The general architecture of the cloud environment 702 depicted in FIG. 7B includes an arrangement of computer hardware and software components that may be used to implement examples of the present disclosure. As illustrated the cloud environment 702 includes one or more hardware processors 708, a remote application manager 710, a registration manager 712, a map server manager 714, a distress notification manager 716, a non-distress manager 718, and an opioid user database 720, all of which may communicate with one another by way of a communication bus. Components of the cloud environment 702 may be physical hardware components or implemented in a virtualized environment. The remote application manager 710, the registration manager 712, the map server manager 714, the distress notification manager 716, and the non-distress 718 manager may include computer instructions that the one or more hardware processors execute in order to implement one or more example processes. The cloud environment 702 may include more or fewer components than those shown in FIG. 7B.

The remote application manager 710 may oversee the monitoring and notifications of associated with the plurality of opioid user systems 706A . . . 706N. The remote application manager 710 is remote in the sense that it is located in a centralized environment as opposed to each opioid user's local environment. The remote application manager 710 may oversee the registration manager 712, the map server manager 714, the distress notification manager 716, and the non-distress notification manager 718. The remote application manager 710 may perform one or more of the steps of FIGS. 2B, 4.

The registration manager 712 may manage the information associated with each opioid user registrant and the contact information supplied by each opioid user registrant during registration for the opioid overdose monitoring system. The contact information may include the names, phone number, email addresses, etc. of individuals and/or organizations to contact on behalf of the opioid user when an overdose event is predicted or detected, or for status check information, as well as the name, address, phone number, email address, etc. of the opioid user registrant. Examples of individuals and organizations are illustrated in FIG. 1B. The opioid user information and the contact information associated with each opioid user registrant may be stored in database 720. FIGS. 5B, 5C illustrate examples of interface screens that may be used during registration.

The map server manager 714 may locate maps and directions, such as those illustrated in FIGS. 3E and 5F to display on devices associated with first responders, friend and family, and other individuals from the opioid user's contact information to display maps or directions to the opioid user, to the location of the closest naloxone or other such medication to the opioid user, and the like, in the event of an overdose. FIGS. 5E, 5F illustrate examples of distress notifications. The map server manager 714 may interface with third party map sites via the network 704 to provide the maps and directions.

The distress notification manager may receive an alert from the opioid user's mobile device that an overdose event may soon occur or has occurred. For example, the mobile device 120 or the monitoring device 110 may process the sensor data from the sensors 102 and determine that an overdose event is occurring. The mobile device 120 may communication the occurrence of overdose event with the distress notification manager 716. The distress notification manager 716 may retrieve contact information from the database 720 and provide notification of the overdose event or a soon to occur overdose event to the individuals and organizations indicated by the opioid user during registration so that assistance can be provided to the opioid user. FIG. 5F illustrates an example of a distress notification.

The non-distress notification manager 714 may receive the status of the opioid user as monitored by the mobile device 120 and/or the monitoring device 110. The non-distress notification manager 718 may receive the status periodically. After determining that the status of the opioid user indicates that the opioid user is not in distress, the non-distress notification manager may access the database 720 to retrieve the contact information for the individual and organizations that are to be notified of the well-being of the opioid user. FIGS. 3B, 3C, 5D illustrate examples of non-distress notifications.

FIG. 7C illustrates an example opioid user system 706, which includes the monitoring device 740 and the mobile communication device 722. The monitoring device can include the sensor(s) 120 that are sensing physiological state of the opioid user and the signal processing device 110 that is processing the raw sensor data from the sensor(s) 110 to provide the mobile communication device 722 with the physiological parameters 118. The raw sensor data 104 from the sensor(s) 102 can be input into the mobile communication device 722, which processes the raw sensor data 104 to provide the physiological parameters 118 of the opioid user.

The illustrated mobile communication device 722 includes a display 724, similar to display 122, described herein, a network interface 726 that is configured to communication at least with the cloud environment 702 via the network 704, a local application 728, a monitoring application 730, a distress application 732, a non-distress application 734, a query opioid user application 736, and a local alarm application 738. The local application 728, the monitoring application 730, the distress application 732, the non-distress application 734, the query opioid user application 736, and the local alarm application 738 may be software instructions stored in memory within the mobile communication device 722 that are executed by the computing devices within the mobile communication device 722. The applications 728-738 can be downloaded onto the mobile communication device 722 from a third party or from the cloud environment 702. The mobile communication device 722 may include more or fewer components than those illustrated in FIG. 7C.

The local application 728 may oversee the communication with the remote monitoring manager of the cloud environment and may oversee the monitoring application 730, the distress application 732, the non-distress application 734, the query opioid user application 736, and the local alarm application 738. The local application 728 is local in the sense that it as well as its associated applications 730-738, are located on the mobile communication device 722 associated with the opioid user, devices associated with organizations to assist opioid users, and devices associated with individuals that are associated with the opioid user.

The monitoring application 730 may receive the physiological parameters 118 and process the physiological parameters according to one or more of the steps of FIGS. 2B, 4. The monitoring application 730 may cause the display of the physiological parameters 118 on the display 724 mobile communication device 722. FIGS. 5A, 5D illustrate examples of displays of the physiological parameters.

The distress application 732 may be called when the monitoring application 730 determines that the opioid user is experiencing an overdose event or an overdose event is imminent. The distress application 732 may perform one or more steps of FIGS. 2B, 4, such as send out distress notifications. Further, the distress application 732 may communicate with the distress notification manager 716 in the cloud environment 702 to cause the distress notification manager to provide distress notifications as described above.

The non-distress application 734 may be called when the monitoring application 730 determines that the opioid user is not experiencing an overdose event or an overdose event is not imminent. The non-distress application 734 may perform one or more steps of FIGS. 2B, 4, such as send status notifications. Further, the non-distress application 734 may communicate with the non-distress notification manager 718 in the cloud environment 702 to cause the non-distress notification manager to provide status notifications as described above.

The query opioid user application 736 may be called when the monitoring application 730 determines that care is indicated. The query opioid user application 736 queries the user to determine whether the user is conscious in order to reduce false alarms. The query opioid user application 736 may perform step 235 of FIG. 2B. FIG. 3A illustrates a display to query the user that may be caused by the query opioid user application 736.

The local alarm application 738 may be called when the monitoring application 730 determines that on-site care of the opioid user is required. The local alarm application 738 may perform step 430 of FIG. 4. The local alarm application 738 may cause the mobile communication device 722 to display first responder instruction, a map, or directions to the nearest facility with medication to reverse or reduce the effects of an overdose, such as naloxone, and the like. The local alarm application 738 may cause the mobile communication device 722 to audibly alarm and/or visually alarm to alert anyone near the mobile communication device 722 of the overdose event. FIG. 3D illustrates an example of a first responder instructions and FIG. 3E illustrates an example of a display displaying the location of naloxone.

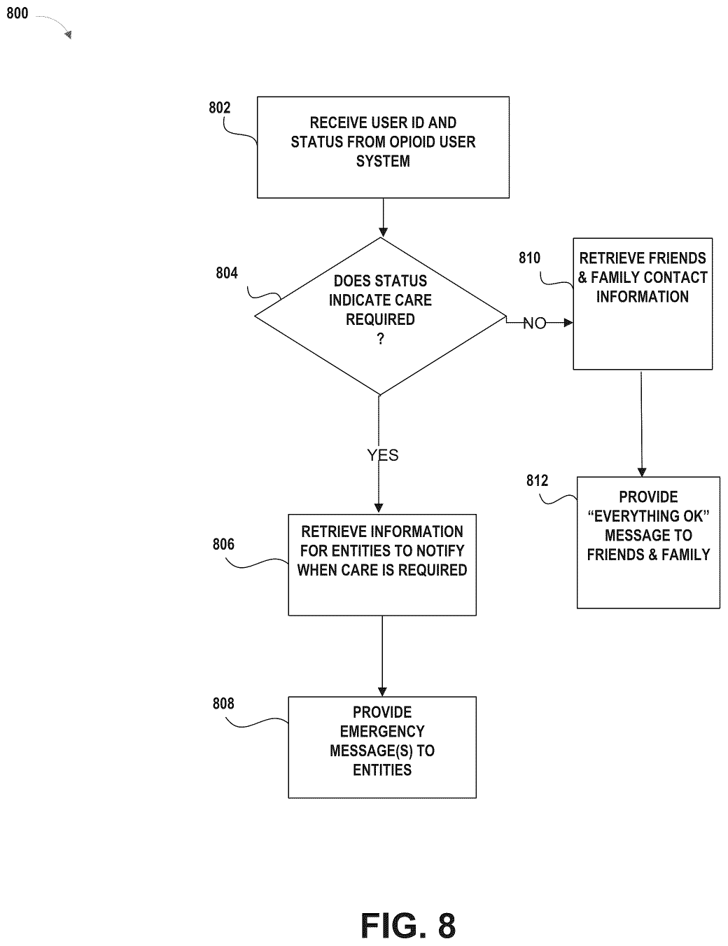

FIG. 8 is a flowchart of an example process 800 to notify an opioid user's notification network of the status of the opioid user. The process 800 can be performed by the cloud environment 702. At block 802, the cloud environment 702 receives a user identification and user status from the opioid monitoring system 706. For example, the remote application manager 710 retrieves the user information from the database 720 based on the user identification.

At block 802, the cloud environment 702 may determine, based on the status of the user, whether care is indicated. The status information may comprise the physiological parameters 118 from the monitoring application 730. The status may be an indication of whether care is indicated or not indicated. Remote application manager 710 may analyze the physiological parameters 118 to determine whether care is indicated.

If care is indicated at block 804, the process 800 moves to block 806. At block 806, the distress notification manager 716 may retrieve the contact information stored in the database and associated with the user identification.

At block 808, the distress notification manager 716 may notify the individuals and organizations of the contact information of the need for care.

If care is not indicated at block 804, the process 800 moves to block 810. At block 810, the non-distress notification manager 718 may retrieve the contact information stored in the database and associated with the user identification.

At block 812, the non-distress notification manager 718 may notify the individuals and organizations of the contact information of the status of the opioid user. The non-distress notification manager 718 can send an “Everything OK” message.

Communication Between Opioid Overdose Monitoring Application and Transportation/Ride Sharing Services

A mobile device or other computing device executing the opioid monitoring application can communicate with one or more transportation services such as, a ride sharing service, such as Lyft® or Uber®, for example, a taxi service, or any commercial transportation service, when an overdose event is occurring or imminent. This is illustrated in FIG. 1B as “Rideshare network” that is within the representation of the location of naloxone message. The opioid monitoring application may communicate, via the mobile computing device, with servers associated with the ridesharing services over a network such as the Internet. The communication can be entered into the transportation service system the same as a person would normally call for a taxi, Lyft, or Uber, for example.

The transportation service can receive a notification from the mobile device or other computing device that is deploying the opioid overdose monitoring application. The notification can be an alert. The alert may be for an ongoing or an imminent opioid overdose event. The notification may include the address of the opioid user, the address of the nearest facility with medication to reverse or reduce the effects of an overdose, such as naloxone, buprenorphine, combination of buprenorphine and naloxone, and the like, and the address of the nearest caregiver, emergency service, treatment center, and other organizations or individuals that can provide life-saving care to for the opioid user.

The transportation service can transport the opioid user to receive care, transport the opioid user to a location having the medication, transport the medication to the opioid user, to pick up the medication and transport the medication to the opioid user, and the like.

The transportation service or ride sharing service can bill for the transportation that occurs after receiving an alert or notification generated by the opioid overdose monitoring application as a special billing or a charitable billing. The transportation service or ride sharing service can bill for the transportation in the same manner that its transportation services are billed for a typical customer.

The transportation service or ride sharing service can participate in a community outreach program to provide transportation responsive to receiving an alert or notification generated by the opioid monitoring application.

### Hub Based Opioid Monitoring System

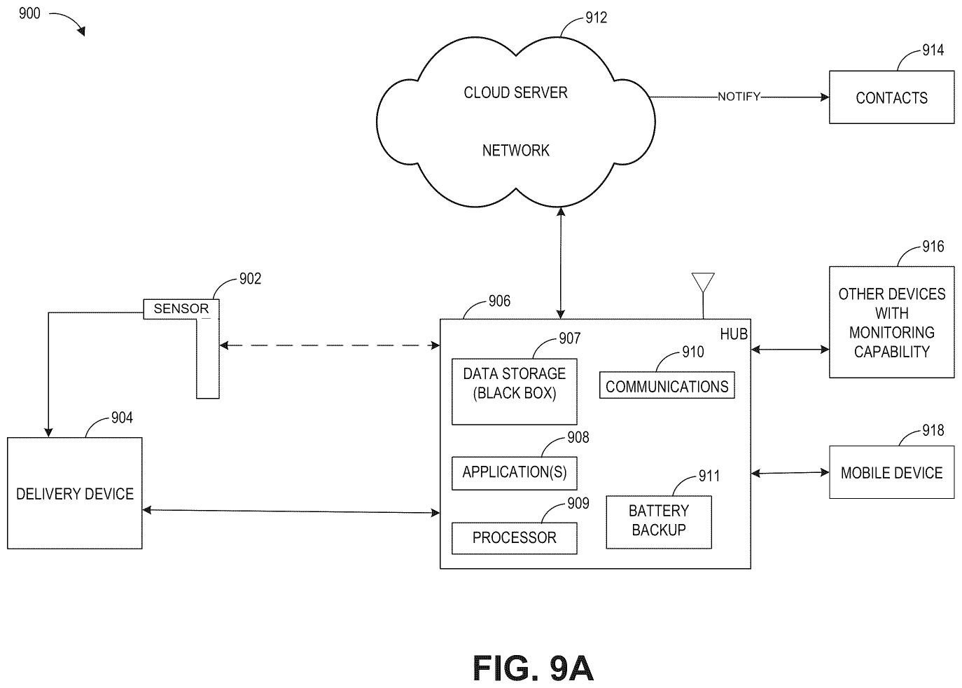

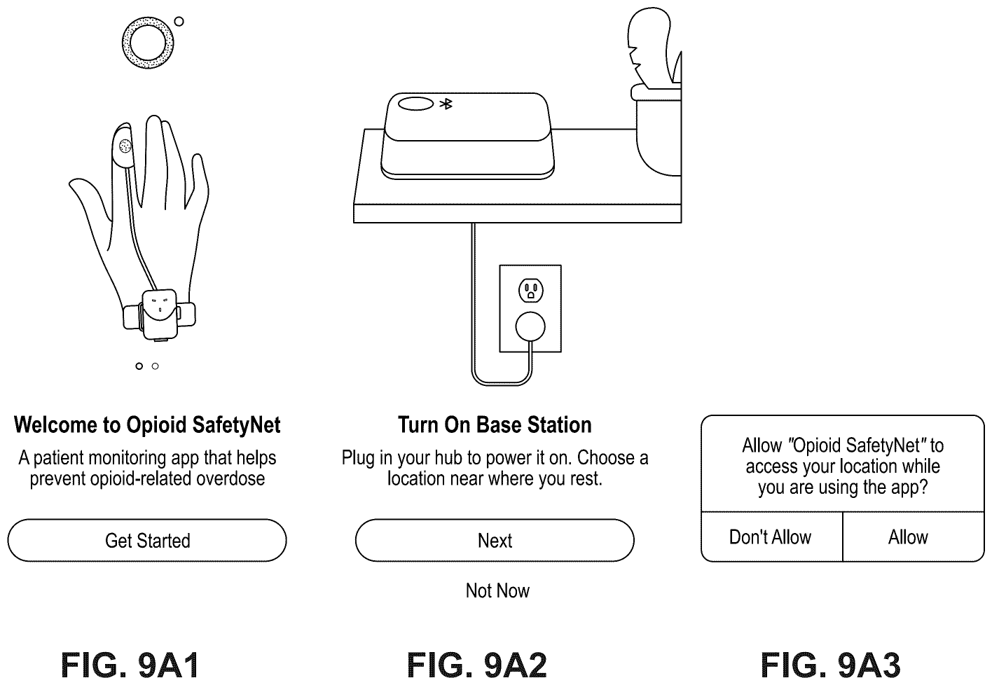

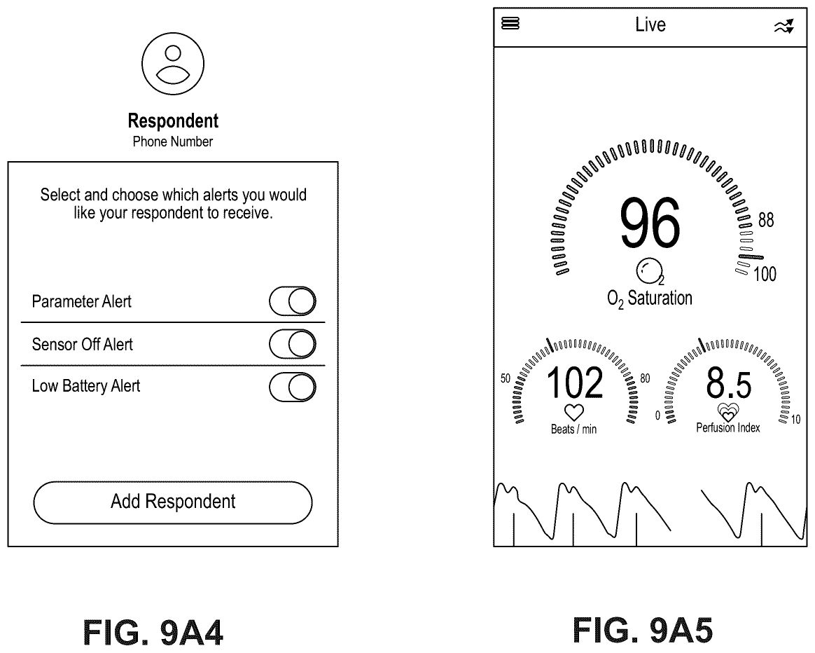

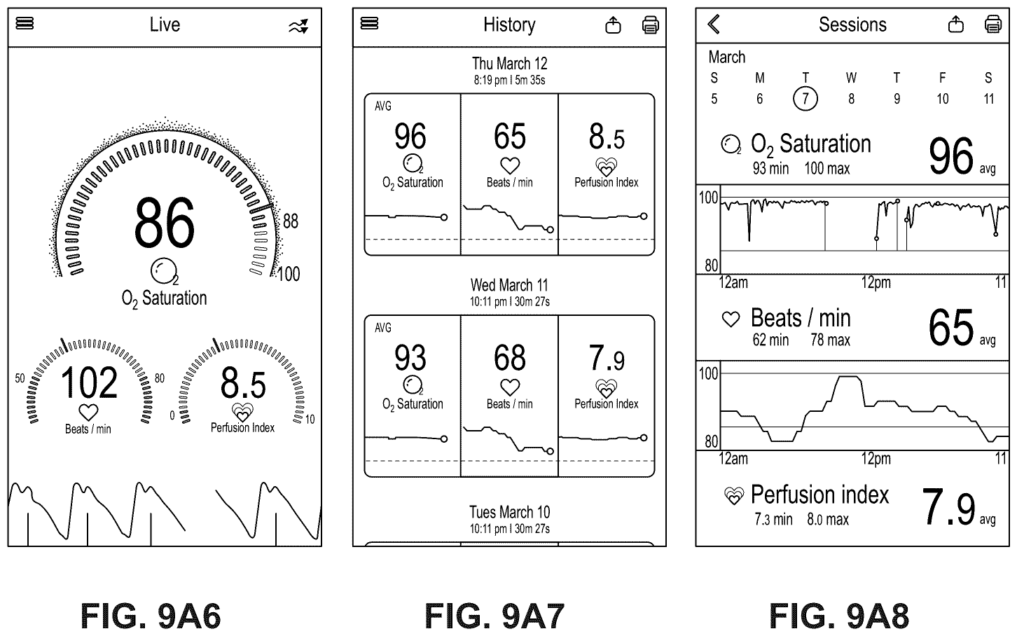

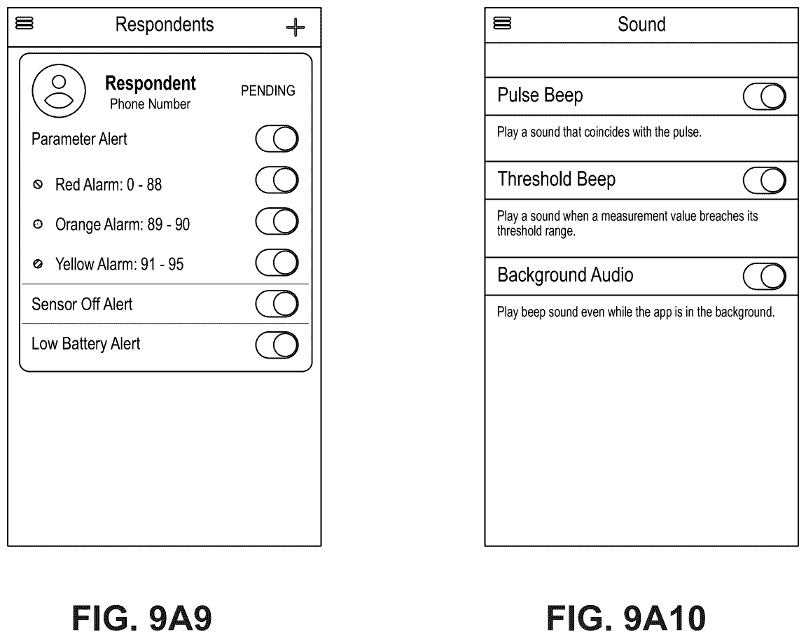

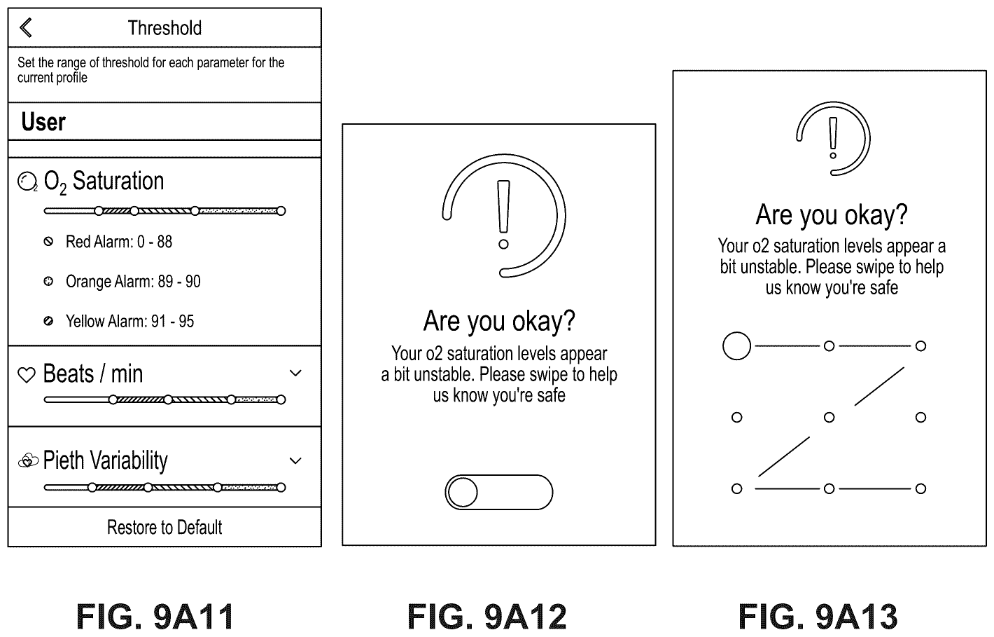

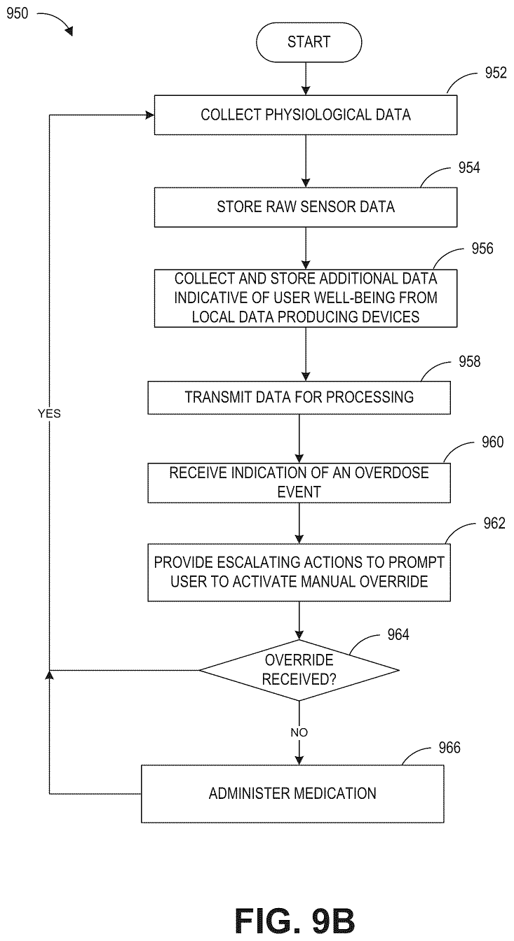

FIG. 9A is a block diagram of an example opioid use monitoring system 900 that includes a sensor 902, a delivery device 904, a medical monitoring hub device 906, and a network 912, such as the Internet hosting a cloud server, which can be considered a remote server because it is remote form the user. Sensor 902 is configured to monitor the user's physiological parameters and deliver device 904 is configured to deliver a dose of an opioid receptor antagonist, such as Naloxone or the like, when an opioid overdose event is detected. Sensor 902 can be an oximetry device, respiration monitor, devices described herein to obtain the user's physiological parameters, and the like. The sensor 902 can be an acoustic sensor, a capnography sensor, or an impedance sensor to monitor the user's respiration rate. The sensor 902 can includes the signal processing device 110 to process the raw sensor data.

Delivery device 904 can be a self-administrating device. The delivery device can be a device that is user or responder activated. The sensor 902 can be internal to the delivery device 904. The sensor 902 can be external to the delivery device 904.

The hub device 906 can be configured to collect data and transmit the data to a cloud server for evaluation. The hub device 906 can comprise communications circuitry and protocols 910 to communication with one or more of the delivery device 904, the sensor 902, network 912, mobile communication device 918, such as a smart phone and the like, and other devices with monitoring capabilities 916. Communications can be Bluetooth or Wi-Fi, for example. The hub device 906 can further comprise memory for data storage 907, memory for application software 908, and a processor 909. The application software can include a reminder to put on the patch before sleeping. The hub device 906 is powered by AC household current and includes battery backup circuitry 918 for operation when the power is out. The hub device 906 can be powered through a USB port, using a charger connected to an AC outlet or connected to an automobiles USB charging port. The hub device 906 can annunciate a battery-low condition.

The hub device 906 can be a Radius-7® by Masimo, Irvine, CA. The hub 906 can comprise at least the memory for data storage 907 and the battery backup circuitry 918 can physically interface and communicate with the Radius-7®. The hub device 906 can interface with the phone cradle of the Radius-7®).

The sensor 902 can monitor the user's physiological parameters and transmit the raw sensor data to the delivery device 904, via wired or wireless communication. Optionally, the sensor 902 can transmit the raw sensor data to the hub device 906, via wired or wireless communication. The delivery device 904 can process the raw sensor data to determine when an opioid overdose event occurs. The hub device 906 can process the raw sensor data to determine when an opioid overdose event occur. The hub device 906 can transmit the raw sensor data to a cloud server for processing to determine when an opioid overdose event occurs. When an opioid overdose event is imminent or occurring, the cloud server can transmit to the delivery device 904 via the hub device 906 instructions to activate and deliver the opioid receptor antagonist, such as Naloxone. The cloud server can further transmit messages to contacts 914, such as friends, family emergency personnel, caregivers, police, ambulance services, other addicts, hospitals, and the like. The hub device 906 can send the delivery device 904 instructions to activate.

It is important to avoid false-positive indications of an overdose event. Users may not wear the self-administrating delivery device 904 if the user experiences delivery of the opioid receptor antagonist when an overdose event is not occurring or imminently going to occur. To avoid false-positive indications, the wearable delivery device 904 can induce pain before administrating the opioid receptor antagonist when an overdose event is detected to inform the user that the antagonist will be administered. The wearable delivery device 904 can provide electric shocks to the user to induce pain. The induced pain can escalate until a threshold is reached. The user can employ a manual override to indicate that the user is conscious and not in need of the opioid receptor antagonist. The override can be a button, switch, or other user input on the delivery device 904, the mobile communication device 722 and/or the hub device 906. The delivery device 904, the mobile communication device 722 and/or the hub device 906 can wait for the user input for a period of time before triggering the release of the opioid receptor antagonist to avoid false-positive indications. The period of time can be less than 1 minute, less than 5 minutes, less than 10 minutes, between 1 minute and 5 minutes, between 1 minute and 10 minutes, and the like.

The memory for data storage 907 can store the raw sensor data. The memory for data storage can act as a “black box” to record data from a plurality of sources. It is critical to administer the opioid receptor antagonist to a user as soon as an opioid overdose event is detected. The opioid overdose event can be cessation of respiration or an indication that respiration will soon cease. The administration can be by a responder, such as a friend or emergency personnel, by a self-administrating device worn by the user, or by the user. To avoid missing any signs that lead to an opioid overdose event, the hub device 906 can receive data from any devices with a monitoring capability. For example, many homes have household cameras which provide a video feed. Cell phones can provide text messages and also include microphones to record voice. The cell phone or smart phone can be configured to listen to breathing and transmit the breathing data. Intelligent personal assistants, such as Amazon's Alexa® controlled Echo speaker, Google's Google Assistant®, Apple's Siri®, and the like, for example, also include microphones and have the ability to interface with the Internet. Many household appliances, such as refrigerators, washing machines, coffee makers, and the like, include Internet of Things technology and are also able to interface with the Internet. Medical monitoring devices that are being used by the opioid user for medical conditions, such as ECG's may also provide additional data. Data from one or more of these devices can be stored in the memory 907 and used by the hub device 1806 or sent to the cloud server and used by the cloud server to detect an opioid overdose event. The hub device 906 can determine what monitoring and Internet-connected devices are available and connect wirelessly to the available monitoring and Internet connected devices to receive data.

The hub device 906 can interface with an internet filter, such as a Circle® internet filter that connects to a home network to monitor content. The hub device 906 can determine which network data is directed to the user's well-being and store the well-being data.

The data can comprise text messages, voice recordings, video, and the like. Because of privacy concerns, the hub device 906 can determine which small portions of data are helpful to determining the user's physical condition and store only those portion of data.

Because devices can fail to connect to the Internet, it is important to have redundant systems to report the sensor data for overdose detection. In the event that the hub device 906 fails to connect to the Internet 912, the mobile device or other internet-connected devices found in the home can provide an internet connection. For example, the hub device 906 can transmit the sensor data to the mobile device 918 and the mobile device 918 can transmit the sensor data to the cloud server for processing. The sensor 902 or delivery device 904 can communicate with the mobile device 918 when the hub device to Internet connection fails. Intelligent personal assistants and IoT devices can also provide redundant (backup) internet communication. The hub device 906 can annunciate when its internet connection fails.

The mobile device 918 can monitor respiration rate, SPO₂, or ECG in parallel with the sensor 902 and hub device 906 monitoring of the user's physiological parameters to increase the likelihood that an imminent overdose will be detected. The sensor 902 can monitor the concentration of an opioid in the user's bloodstream. The measured concentration can be a factor in determining an opioid overdose event to reduce instances of false positives.

A home security monitoring system can include the hub device 906 and a home security company can monitor the user's health via the hub device 906 and sensor 902.

The opioid overdose monitoring application can be integrated into intelligent personal assistants, such as Amazon's Alexa®, for example.

The delivery device 904 can include medication to induce vomiting. The opioid user can ingest the vomit-inducing medication, if desired, to regurgitate any opioid substance remaining in the user's stomach. The delivery device 904 can include reservoirs containing the vomit-inducing medication and a position-sensing sensor. The vomit-inducing medication can be automatically dispensed after receiving sensor input indicating that the user is in an upright position.

The position-sensing sensor can monitor the user's movements to determine that the user is upright. The delivery device 904 can include one or more sensors configured to obtain position, orientation, and motion information from the user. The one or more sensors can include an accelerometer, a gyroscope, and a magnetometer, which are configured to determine the user's position and orientation in three-dimensional space. The delivery device 904 or the hub device 906 can be configured to process the received information to determine the position of the user.

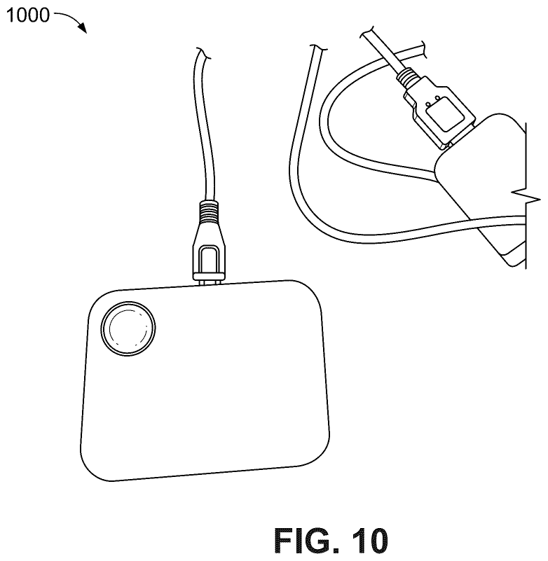

FIG. 10 illustrates an example hub device 1000 of the opioid overdose monitoring system of FIG. 9A. FIG. 9B is a flow diagram of a process 950 to administer the opioid receptor antagonist using the system of FIG. 9A. At block 952, the sensor 902 can collect raw sensor data that comprises physiological data. The sensor 902 can transmit the raw sensor data to the delivery device 904 and the delivery device 904 can transmit the raw sensor data to the hub device 906. Alternately, the sensor 902 can transmit the raw sensor data to the hub device 906.

At block 954, the hub device 906 can store the raw sensor data. At block 956, the hub device 906 can collect and store data associated with the user's well-being from other devices local to the user. For example, the hub device can receive data from one or more home cameras, data from microphones and cameras of intelligent home assistants, such as Alexa®, for example, internet data from a home internet filter, and the like.

At block 958, the hub device 906 can transmit via the network 912, the stored data to a cloud server for processing. The cloud server can process the data to determine whether an opioid overdose event is occurring or will be imminent. At block 960, the hub device 906 can receive from the cloud server an indication that an opioid overdose event is occurring or imminent. The hub device 906 can transmit the indication to the delivery device 904.

At block 962, the delivery device 904 can provide the user with escalating actions to prompt the user to activate a manual override to indicate that the opioid overdose event is not occurring. For example, the delivery device can provide increasing electric shocks to the user, up to a threshold.

At block 964, the delivery device 904 can determine whether an override from the user has been received. When an override is indicated, such as from a user activated button or switch on the delivery device 904, the process 950 returns to block 952 to continue collecting physiological parameters. When an override is not indicated, the process 950 moves to block 966. At block 966, the delivery device 904 administers the medication, such as Naloxone or other opioid receptor antagonist and returns to block 952 to continue monitoring the physiological parameters.

FIGS. 9A1-9A13 illustrate various example software applications to trigger an alarm and notify a friend when an opioid overdose is indicated. The software application can be downloaded onto the user's smart mobile device 918.

FIG. 9A1 is an example screenshot illustrating a welcome message to a new user of the opioid overdose monitoring application. The illustrated screenshot of FIG. 9A1 displays an illustration of a hand wearing an example sensor and signal processing device 902. The user can create an account for the overdose monitoring application. Once account registration is successful, the example application 908 can instruct the user to set up the communications between the mobile device 918, the sensor and signal processing device 902, the medical monitoring hub device 906, and the home Wi-Fi network.

FIG. 9A2 is an example screenshot illustrating instructions to the user to power the medical monitoring hub device 906 to wireless connect to the mobile device 918. For example, the medical monitoring hub device 906 can be Bluetooth enabled.

FIG. 9A3 is an example screenshot asking the user to allow the software application to access location information. When the software application has access to the user's location information such as the location information found on the user's mobile device 918, the software application can provide the user's location to emergency personnel, caregivers, friends, and family, etc. when they are notified of an overdose event.

FIG. 9A4 is an example screenshot illustrating a selected respondent to be notified in the event of an opioid overdose event, where the opioid overdose event can be an overdose that is presently occurring or, based on the user's physiological parameters sensed by the sensor and signal processing device 902, will soon occur. The selected respondent can also be notified of situations that may cause the opioid monitoring system to fail if not corrected, such as when the user is not wearing the sensor or the sensor battery is low. The illustrated screenshot of FIG. 9A4 displays the selected respondent's name and phone number and provides a selection of alerts that the user can choose the respondent to receive. The example selections include a parameter alert, a sensor off alert, and a battery low alert. The parameter alert can be sent when the monitored physiological parameter falls outside a range of acceptable values. The sensor off alert can be sent when the user is not wearing the sensor and signal processing device 902. The batter low alert can be sent when the battery voltage in the sensor and signal processing device 902 fall below a threshold value.

FIGS. 9A5-9A6 are example screenshots illustrating the real time monitoring of the user's physiological parameters. The illustrated screenshots of FIGS. 9A5-9A6 display representation of dials indicating the monitored oxygen saturation, heart rate in beats per minute, and perfusion index. The illustrated screenshot of FIG. 9A5 indicates that the monitored oxygen saturation (96), heart rate (102), and perfusion index (8.5) are acceptable values. The illustrated screenshot of FIG. 9A6 indicates that the monitored oxygen saturation (86) is no longer within an acceptable range.

FIG. 9A7 is an example screenshot illustrating historical averages of the user's monitored physiological parameters. The illustrated screenshot of FIG. 9A7 displays the average oxygen saturation, heart rate, and perfusion index for the period of time the sensor and signal processing device 902 collected data for two dates, March 11, and March 12.

FIG. 9A8 is an example screenshot illustrating session data for oxygen saturation, heart rate, and perfusion index on March 7. The displayed information in the illustrated example includes the minimum, maximum and average of the monitored physiological parameter.

FIG. 9A9 is an example screenshot illustrating a selection of parameter notifications to be sent to the selected respondent. In the illustrated screenshot of FIG. 9A9, the user can select to send the respondent any combination of a red alarm, an orange alarm, and a yellow alarm. For example, for the oxygen saturation parameter, a red alarm can be sent when the user's oxygen saturation falls within the range of 0-88; an orange alarm can be sent when the user's oxygen saturation falls within the range of 89-90, and a yellow alarm can be sent when the user's oxygen saturation falls within the range of 91-95 to provide an indication of the severity of the overdose event to the respondent.

FIG. 9A10 is an example screenshot illustrating sound options available for the software application. In the illustrated screenshot of FIG. 9A10, the software application can cause the mobile device 918 to play a sound, such as a beep, that coincides with the user's pulse, play a sound, such as a beep, when a measurement value breaches its threshold range, and play a beep sound even when the software application is running in the background.

FIG. 9A11 is an example screenshot illustrating customizable alarm values. Some users may have a higher tolerance for opioids and an opioid event may not be occurring when the user's physiological parameters fall within a range that typically signals an opioid overdose event. It is desirable to avoid false alarms that may desensitize respondents to notifications. In the illustrated screenshot of FIG. 9A11, the ranges for a red, orange, and yellow alarms for oxygen saturation can be customized for the user by, for example, sliding the indicators along the green-yellow-orange-red bar until the desired values are displayed. Selecting beats/minute and pleth variability permits the user to customize the alarm ranges for heart rate and perfusion index, respectively.

FIGS. 9A12-9A13 are example screenshots illustrating a request for user input when the user's physiological parameters indicate an opioid overdose event is occurring or will soon occur. To avoid sending false alarms, the software application requests user input to confirm that the user is not unconscious or otherwise does not want alarm notifications to be send to respondents. In the illustrated screenshot of FIG. 9A12, the user is asked to swipe the screen to confirm safety. In the illustrated screenshot of FIG. 9A13, the user is asked to enter an illustrated pattern on the screen to confirm safety. Different user inputs can be used to confirm different cognitive abilities of the user. For example, it is more difficult to enter the illustrated pattern of FIG. 9A13 than to swipe the bottom of the screen in FIG. 9A12.

### Reducing False Positive Reporting

False positive reporting of an opioid overdose event will cause the recipients, such as the user, friends, and family, skeptical that an overdose event is actually occurring and they may not take the appropriate action in the event of on actual opioid overdose event.

### Critical Time-Based Opioid Monitoring

Critical time-based opioid monitoring involves identifying best data in the first few minutes after taking an opioid drug to reduce false reporting of an opioid overdose event. Monitoring is based on physiological parameter monitored by a physiological parameter monitoring assembly. The physiological monitoring can use a pulse oximeter that includes a sensor and a signal processing device. Examples of physiological parameters that can be monitored are peripheral oxygen saturation (SpO₂), respiration, and perfusion index (PI). The application can determine the physiological condition of the user based on the SpO₂ alone, respiration alone, PI alone, a combination of the SpO₂ and respiration, a combination of the SpO₂ and PI, a combination of the respiration and the PI, or a combination of the SpO₂, respiration, and PI. Critical time periods for monitoring the user's physiological parameters for an indication of an opioid overdose event can be within a period of time immediately following the use of the opioid drug. Examples can be within 20 minutes from the time of drug use, less than 20 minutes from the time of drug use, or more than 20 minutes from the time of drug use. Continuous monitoring for a period of time after drug use, such as the first 20 or 30 minutes after drug use, can be monitored for indications of an opioid overdose event. Other periods of time can be monitored. Other critical times to monitor the user's response to drug use can be a particular time of day, before sleeping, or during the day.

### Body Modeling

The opioid monitoring device, systems, and methods described here can monitor physiological parameters of the user. Some non-limiting examples of the physiological parameters that can be monitored are peripheral oxygen saturation (SpO₂), respiration, and perfusion index (PI). The application can determine the physiological condition of the user based on the SpO₂ alone, respiration alone, PI alone, a combination of the SpO₂ and respiration, a combination of the SpO₂ and PI, a combination of the respiration and the PI, or a combination of the SpO₂, respiration, and PI. Over time, the device, such as the smart device, or hub device, or server, described herein, can learn the typical ranges of an individual's monitored physiological parameters. The device can create a transfer function for the user's body and determine when a monitored physiological is greater than or less than a threshold value of the monitored physiological parameter. Deviating from a threshold value of the monitored parameter can provide an indication of an opioid overdose event. In another example, the body transfer function and the monitored physiological parameter can provide a check to reduce or eliminate false positive indications of an opioid overdose event. For example, a specific monitored parameter may have a value that for an average person would indicate an overdose has occurred or will soon be occurring. The body transfer function for the individual may indicate that the physiological parameter is within a non-overdose condition for that individual.

The device, such as the smart device, the hub, or the server can be an artificial intelligence device by continuously feeding back the monitored physiological parameters to the program that is creating the body transfer function. The artificial intelligence program revises and updates the body transfer model for increased accuracy. In an embodiment, the learned body transfer function may predict drug ingestion. The body transfer function may use parameters across populations, such as those populations associated with the user, and modify those parameters for use in the body transfer function for an individual based on the individual's physiological data. In another example, the body transfer function can use data associated with the monitored parameters that is identified as occurring just prior to an opioid overdose event to update the body transfer function. The updated body transfer function can be finely tuned to predict an opioid overdose event. The body transfer function uses variability in the respiration rate, variability in the heart rate, pulse transit time, hydration, and pleth shape analysis to model the response of the user. Pleth shape analysis provide an indication of vascular tone shape.

### Opioid Overdose Risk Engine and Level of Alarm

One of the physiological responses of opioid toxicity is respiration depression. Others may include disturbances in pulse rate and perfusion index. If left unchecked, respiration may fall below a critical level and, if not corrected, death may result. Oximetry can be used to detect depressed breathing. Oximetry utilizes a noninvasive optical sensor to measure physiological parameters of a person. In general, the sensor has light emitting diodes (LEDs) that transmit optical radiation into a tissue site and a detector that responds to the intensity of the optical radiation after absorption (e.g., by transmission or transflectance) by, for example, pulsatile arterial blood flowing within the tissue site. Based on this response, a processor can determine measurements for peripheral oxygen saturation (SpO₂), which is an estimate of the percentage of oxygen bound to hemoglobin in the blood, pulse rate, plethysmograph waveforms, which indicate changes in the volume of arterial blood with each pulse beat, and perfusion quality index (e.g., an index that quantifies pulse strength at the sensor site), among many others.

In some aspects, an indication of depressed breathing can be a gold standard that is used to determine an overdose event. However, in some instances, an indication of depressed breathing may not be clinically significant if there is a disturbance in the physiological parameters. Notifications based on disturbances in physiological parameters that are not clinically significant can result in false positive notifications. Notifications based on transient measurements of the physiological parameters can result in false positive notifications. In a home setting, without professional monitoring, if a user is notified of an opioid overdose event that is a false positive notification, the user may forgo any monitoring that is designed to ensure the user's well-being. To increase the accuracy of determining whether an opioid overdose event is occurring or will soon occur in a home setting, not in a hospital or other care assisted setting, a risk score determination engine can be used to determine an output based on one or more weighted physiological parameters. The output of the risk score determination engine can be a risk score or a wellness index. In other aspects, other physiological parameters can be used. Examples of the weighted physiological parameters are peripheral oxygen saturation (SpO₂), pulse rate (PR) and perfusion quality index (PI). An example of the risk score determination engine can be Halo ION™ by Masimo Corp. and an example of the risk score can be the Halo Index™. Example calculations of risk score can be found in Halo: Assessing Global Patient Status with the Halo Index™ and hereby incorporated herein by reference in its entirety and appended as Appendix A and U.S. Application No. 10,332,630, filed Feb. 13, 2012, titled Medical Characterization System, assigned to Masimo Corporation, Irvine Corporation (“Masimo”) and hereby incorporated herein by reference in its entirety.

The risk determination engine can weigh and aggregate multiple physiological parameters and the history of these monitored parameters to determine a risk score. The risk determination engine can weigh and aggregate multiple physiological parameters to determine a risk score based on a history of the monitored parameter. The risk score can be used to determine the level of response that is needed. To distinguish between the severities of the physiological parameters, the risk score determination engine can further correlate the trends of multiple physiological parameters. The correlation or pattern matching between multiple physiological parameters can be weighted and included in the risk score processing algorithm. Correlating two physiological parameters can be considered a two-dimensional view (2D) of the user data. Correlating two or more physiological parameters and including the weighted correlation to the risk determination engine increases the accuracy and provides fewer false positive alarms.

Continuous monitoring through the risk determination engine, an advanced pattern recognition algorithm helps detect and quantify the risk of severe opioid-induced respiratory depression. The risk determination engine helps to manage and send escalating alarms to family members, friends, and caregivers, notifying them that help may be needed due to an opioid overdose-including triggering an automatic wellness call, which may lead to EMS being dispatched. The risk score determination engine can be implemented to process escalating alarm levels in parallel. The risk scores and/or alarm can be used to determine a treatment or improving a treatment for a user. Each alarm level provides a different level of intervention. For example, a risk or wellness score that indicates a level 1 alarm can indicate a local rescue. Examples of a local rescue include providing an audible alarm to wake up the user, requesting user input to indicate consciousness. A risk or wellness score that indicates a level 2 alarm can initiate an intermediate rescue, which is escalated from the local rescue. An intermediate rescue can indicate that another person, other than the user, or stimulation, other than sound, may be needed provide intervention. An example of an intermediate rescue can be sending a message to a friend or family member that the user has previously designated. Another example of an intermediate rescue can be providing physical stimulation to the user. The provided physical stimulation can be physically uncomfortable in order to wake up the user. A risk or wellness score that indicates a level 3 alarm can escalate a response beyond that of a level 2 alarm. Examples of the response to a level 3 alarm can be initiating professional assistance, such as notifying paramedics to respond with an opioid receptor antagonist (i.e., Naloxone or Narcan). Accordingly, the risk score and/or alarms can be used to provide treatment for a user.

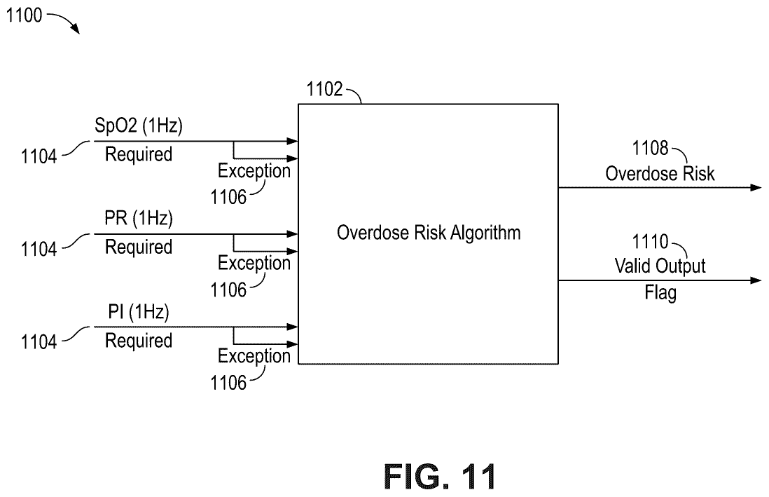

FIG. 11 illustrates a block diagram 1100 of an example risk score determination process and system for measured physiological parameters. The system can comprise a controller 1102 that executes a set of software instructions to perform the risk algorithm. The controller 1102 may take as input, for example, a data stream 1104 for each parameter (SpO₂, PR, PI) and their exception status 1106. Other or fewer parameters and their exception status may also be used. For example, in some examples, temperature or other parameter data stream may be taken as input by the controller 1102 The exception status 1106 can be set by the user and/or a medical professional. In some examples, the exception status 1106 can be set to ignore or discard specific values or periods from the data stream when determining the overdose risk 1108 (which may also be referred to herein as an “OD risk”). In other examples, the exception status 1106 can be set to include values of the data stream during certain events. The exception status 1106 can also be related to confidence in measurements and whether signal noise is disturbing the data streams.

The controller 1102 may output the overdose risk score 1108 and an indicator flag 1110 that the output is valid. The overdose risk score, in these examples, are based on the user's SpO₂, PR, and PI data streams 1104. For producing an overdose risk score 1108, the controller 1102 may require only a subset of parameters, such as SpO₂ and pulse rate (PR). In some examples, one or more parameters may be optionally analyzed by the controller 1102 to produce an overdose risk score. If a parameter is unavailable or determined by the controller 1102 to be an unreliable or incorrect data stream or value, the controller 1102 may determine not to use the unavailable, unreliable, or incorrect data stream or parameter. For example, respiration rate from the pleth (RRp) may be optional and used if available or not used if not available.

The process may treat each stream separately and calculates a score for each one in parallel. In some examples, only the SpO₂ parameter is used for the calculation of the significant desaturation events. Accordingly, the desaturation event related features of the other two parameters are dependent on the detected SpO₂ area. However, there may be common features for all the parameters, such as baseline risk and instability. The calculation of the event related features is described in further detail below. In some examples, the controller 1102 may determine a presence and/or severity of physiological events based on an SpO₂ area, where

$$Area = \frac{sum ( abs ( SpO 2 Threshold - X ) )}{C} , C = 2500 , {SpO}_{2} Threshold = LowerLimit ,$$

X is the SpO₂ value less than the threshold and not zero/invalid and C is a normalization constant.

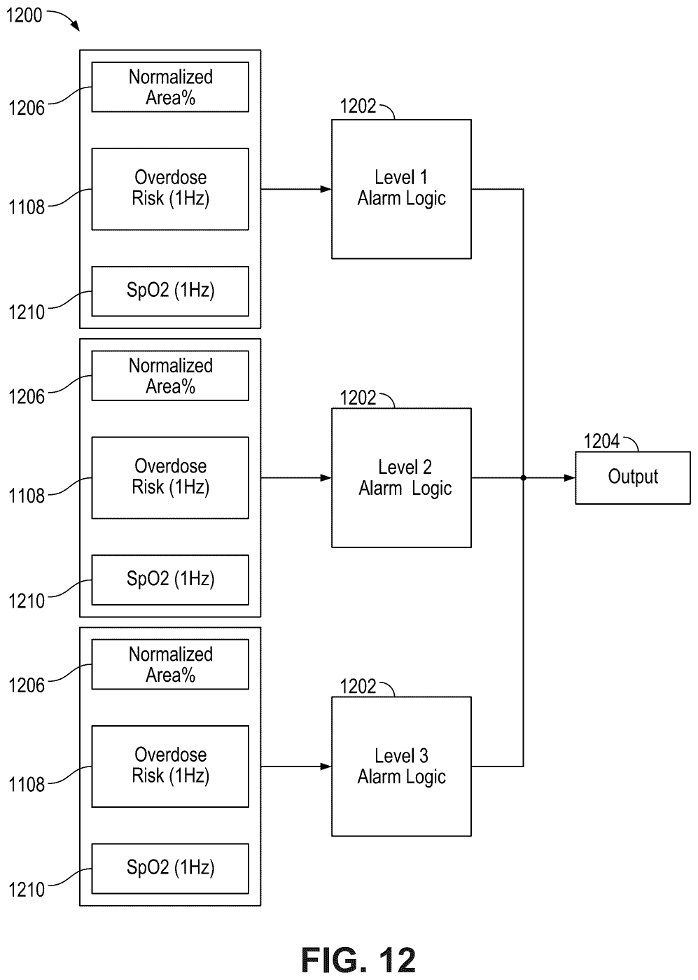

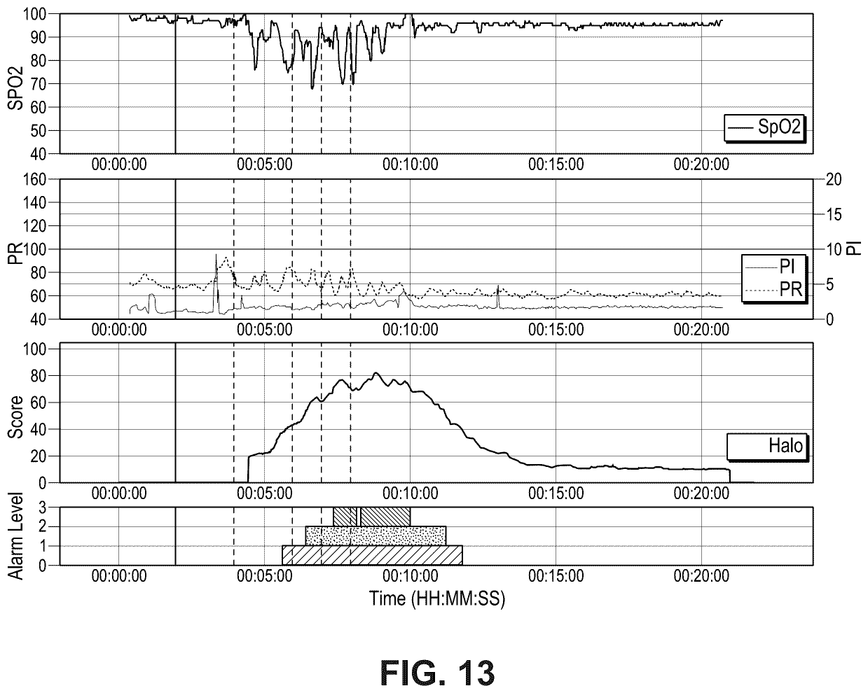

FIG. 12 illustrates a block diagram 1200 of an example alarm level determination. Referring to FIG. 12, the risk score output 1108 from the risk determination engine illustrated in FIG. 11 can be processed by the alarm level logic 1202. In the illustrated embodiment, three alarm levels are processed in parallel. The output 1204 of the alarm processing is a determination of which alarm level is indicated by the processing of the user's physiological parameters by the risk determination engine. Each alarm level is characterized by values of the OD risk 1108, normalized area 1206, and SpO₂ 1210. To generate the normalized area 1206, SpO₂ can be monitored for any drops below a lower limit. The lower limit may be predetermined or set by a user and/or a care provider. In some instances, the lower limit is 85 as shown in the first graph of FIG. 13. The lower limit can also be 90. In some instances, the lower limit may depend on other physiological parameters or condition of a user. As an example, as the SpO₂ level drops below 85, the area of the curve during the duration of the drop is measured until SpO₂ returns to 85. Then, the area of the curve can be normalized as a percentage to generate the normalized area 1206.

In some examples, there can be more than one condition that triggers an alarm level. For example and as shown in Table 1, the OD risk and normalized area can be greater than a first threshold, or the OD risk alone can be greater than a second threshold, or the SpO₂ can be less than a third threshold, each with a time condition. The fourth graph of FIG. 13 illustrates the escalating alarm levels. In the illustrated example. The conditions indicated by the parameter graphs result in the risk score illustrated the score graph. The alarm level graph illustrates that as the score increases, the alarm escalates. The level of intervention indicated by the uppermost bar is greater that the level of intervention indicated by the middle bar, which is greater than the level of intervention indicated by the lowest bar. In some examples and as illustrated in Table 1, there can be more than one condition that triggers an alarm level. For example, the OD risk and normalized area can be greater than a first threshold, or the OD risk alone can be greater than a second threshold, or the SpO₂ can be less than a third threshold, each with a time condition.

TABLE 1

| Notifications | |
|---|---|
| Definition | Criteria |
| Level 1 | Overdose Risk ≥40% and NormalizedArea >0.5%, ≥1 |
| | second or Overdose Risk ≥64%, ≥1 second |
| | or |
| | SpO₂ <85%, ≥30 second |
| Level 2 | Overdose Risk ≥50% and NormalizedArea >2.5%, ≥1 |
| | seconds or Overdose Risk ≥65%, ≥1 seconds |
| | or |
| | SpO₂ <80%, ≥30 seconds |
| Level 3 | Overdose Risk ≥70%, ≥1 seconds |
| | or |
| | SpO₂ <80%, ≥180 seconds or |
| | SpO₂ <60%, ≥60 seconds |

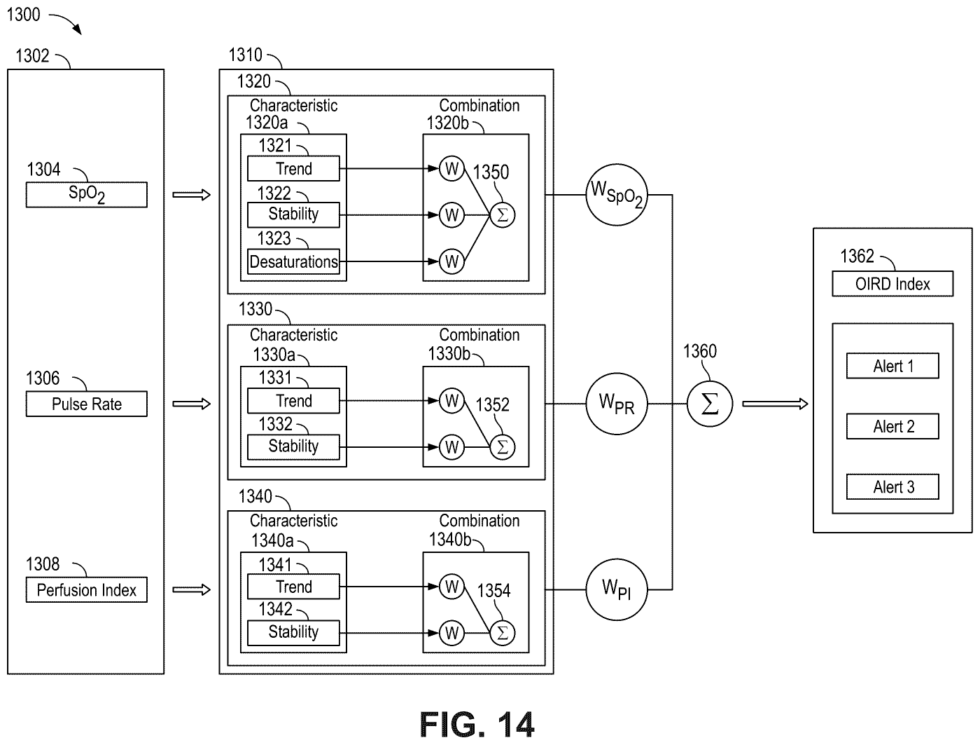

FIG. 14 illustrates an implementation of a block diagram 1300 of example processing that may be performed on example physiological parameter data 1302 including one or more parameters, such as Oxygen Saturation (SpO₂) 1304, Pulse Rate (PR) 1306, and/or Perfusion Index (PI) 1308, as measured by a physiological sensor, to determine a risk score 1360 (i.e., Halo Index) of a user. The block diagram 1300 may include (1) monitoring one or more parameters, (2) extracting one or more characteristics from the one or more parameters, and (3) calculating a risk score. To determine the risk score 1360 (also mentioned herein as an “index score”), the physiological parameters 1302 can be continuously analyzed over relevant time periods in order to extract parameter characteristics. For example, a physiological sensor may detect physiological parameter data 1302 periodically, such as at every second or approximately every second. Some characteristics may apply to multiple parameters whereas others may apply to a specific parameter. The parameter characteristics may include historical parameter data and parameter relationships that may be used to produce an integrated and/or cumulative assessment of a user's underlying physiology along with instantaneous values.

The monitored one or more physiological parameter data 1302 may then be processed by a system 1310 for determining the characteristics of the signal and/or output an overall parameter assessment summarization for each of the parameter characteristics. The system 1310 may be subdivided into one or more subsystems based on the physiological parameter to be processed. For example, SpO₂ 1304 may be inputted into subsystem 1320, PR 1306 may be inputted into subsystem 1330, and PI 1308 may be inputted into subsystem 1340. Each of the subsystems 1320, 1330, 1340 may then be further subdivided for further processing in order to determine the parameter characteristics and/or calculating a summation of the parameter characteristics. A weighting can then be applied to each of the parameter characteristics to determine a parameter assessment value.

The system 1320 for processing SpO₂ 1304 can include a subsystem 1320a for determining parameter characteristics of SpO₂ 1304 and/or subsystem 1320b for calculating a summation of the parameter characteristics of SpO₂ 1304 in which a weighting in applied to each of the parameter characteristics before being combined into a risk assessment.

The system 1330 for processing PR 1306 can include a subsystem 1330a for determining parameter characteristics of PR 1306 and/or subsystem 1330b for calculating a summation of the parameter characteristics PR 1306 in which a weighting in applied to each of the parameter characteristics before being combined into a risk assessment.

The system 1340 for processing PI 1308 can include a subsystem 1340a for determining parameter characteristics of PI 1308 and/or subsystem 1340b for calculating a summation of the parameter characteristics of PI 1308 in which a weighting in applied to each of the parameter characteristics before being combined into a risk assessment.

The combinations of parameter characteristics from subsystem 1320a, subsystem 1330a, and 1340a may then be combined to produce an SpO₂ risk assessment 1350, a PR risk assessment 1352, and/or a PI risk assessment 1354, respectively. The SpO₂ risk assessment 1350, PR risk assessment 1352, and/or PI risk assessment 1354 are then weighted accordingly as compared to other parameters before being combined into a risk score 1360 that provides a cumulative trending assessment of a global user status. The overall parameter assessment is the weighted sum of each of its combined characteristics. Referring to FIG. 14, the weighted values may be applied when calculated the risk assessments 1350, 1352, and/or 1354. In some embodiments, the weightings can be personalized and can change over time based on the user's physiological condition. The weightings can consider parameters such as the user's age, prior health conditions, overall health, etc.

Once the SpO₂ risk assessment 1350, PR risk assessment 1352, and/or PI risk assessment 1354 are determined, the risk score 1360 may be calculated. The risk score 1360 can be the weighed accumulation of the individual parameter assessments 1350, 1352, and/or 1354. For example, the risk score can be expressed as:

$$Risk Score = {W}_{SpO 2} * SpO {2}_{Risk} + {W}_{PR} * {PR}_{Risk} + {W}_{PI} * {PI}_{Risk}$$

The risk score 1360 may then be inputted into an Opioid-Induced Respiratory Depression (OIRD) Index 1362 containing a range of values. For example, the range of values may be between 0 to 1000, between 0 to 750, between 0 to 500, between 0 to 250, between 0 to 200, between 0 to 150, between 0 to 100, between, 1 to 50, between 1 to 20, between 1 to 10, etc. Increases in the risk score 1360 may correspond with physiological deterioration and/or may indicate a need for additional assessments. The risk score 1360 may also be processed by an alarm level determination processes, similar to those described in FIGS. 11 and 12. The one or more alarm levels may be processed in parallel. Depending on the severity of the risk score 1360, a corresponding alarm level may be indicated corresponding to the level of intervention needed. For example, the intervention associated with the determined alarm level can indicate a local rescue. The local rescue can generate an audible alarm. The intervention associated with the determined alarm level can initiate an intermediate rescue. The intermediate rescue can transmit wirelessly a notification to one or more recipients. The intermediate rescue can stimulate the user physically. The intervention associated with the determined alarm level can initiate professional assistance. The professional assistance notifies medical personnel to respond with an opioid receptor antagonist.

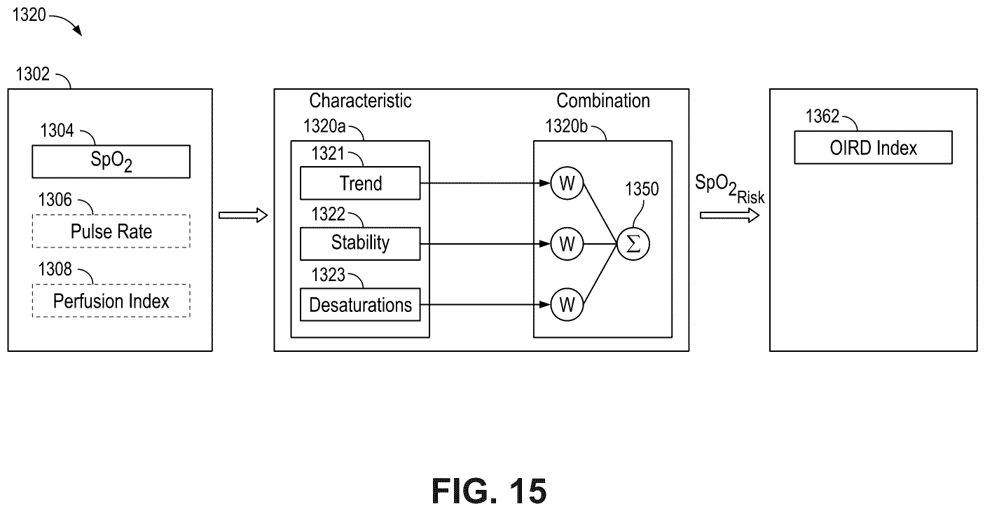

FIG. 15 illustrates an example implementation of the system 1320 for determining the characteristics of the SpO₂ physiological parameter 1304. System 1320 may include the characteristic sub-system 1320a to record and/or identify trend characteristics 1321, stability characteristics 1322, and/or desaturation characteristics 1323 associated with the SpO₂ parameter 1304. The trend characteristic 1321 may track and/or record a user's SpO₂ parameter 1304 over time. For example, the trend characteristic 1321 may include average values recorded over a period of 10 minutes, over a period of 15 minutes, over a period of 30 minutes, over a period of 60 minutes, of a period between 1 minute to 60 minutes, of a period between 5 minutes to 45 minutes, of a period between 7 minutes to 15 minutes, or other periods of time. The trend characteristic 1321 may include historical parameter data and/or parameter relationships related to other monitored parameters. The stability characteristics 1322 may include measurements of the degree to which the instantaneous parameter values corresponding to SpO₂ tend to deviate from their baseline, where large and/or frequent deviations are indicative of high parameter instability. As the opioid limits the ability of the user to breath due to a reduction in the density to carbons dioxide and blocks the user's cardiac responses, oscillations within the SpO₂ parameter may increase as the user struggles to inhale oxygen. The stability characteristic 1322 can include using the historical parameter data to produce smooth, slow changing measurements that convey information of the underlying trends in patient status. The desaturation pressure data 1323 can measure the downward movement of the instantaneous SpO₂ parameter 1304 and record occurrences of low blood oxygen saturation in a user. The downward movement may show an instability within the SpO₂ 1304 measurements, an inability to maintain a normal oxygen saturation, and/or the downward movement may be a precursor to a significant and prolonged drop in saturation.

In the combination system 1320b of system 1320, each of the parameter characteristic values from the parameter characteristic subsystem 1320a can be given a weight based on the degree of abnormality and then combined to output the SpO₂ risk assessment 1350. The weighting may be unique to each user and may also change over time in a patient based at least on their physiological condition.

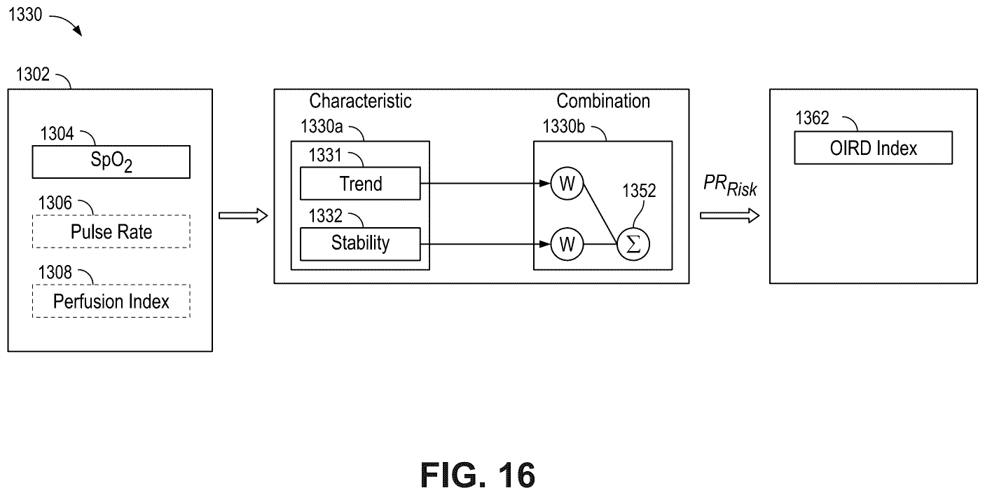

FIG. 16 illustrates an example embodiment of the system 1330 for determining the characteristics of the PR physiological parameter 1306. System 1330 may be similar or identical to system 1302 in some or many respects. With reference to FIG. 16, system 1330 may include a characteristic subsystem 1330a to record and/or identify trend characteristics 1331 and stability characteristics 1332 associated with the PR parameter 1306 and a combination system 1330b calculating a weighed aggregate of the values from the parameter characteristic subsystem 1330a to determine the PR risk assessment 1352.

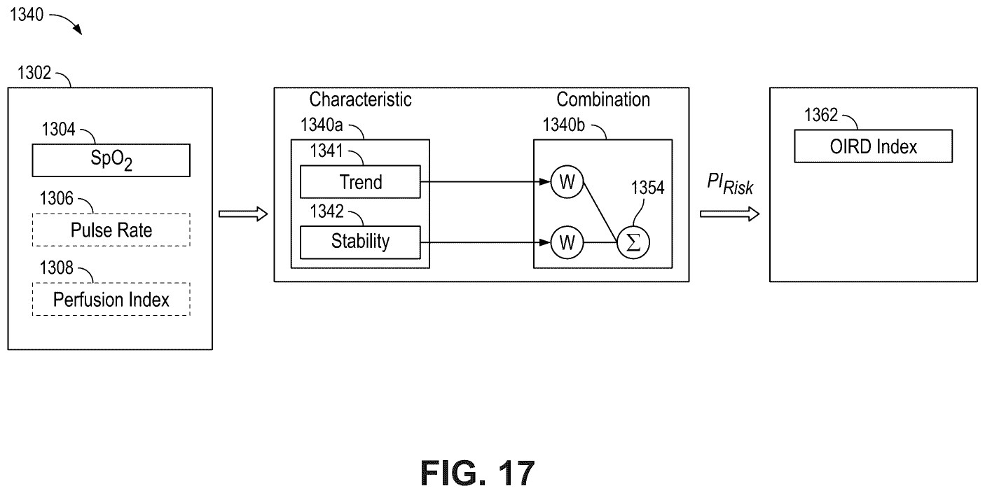

FIG. 17 illustrates an example embodiment of the system 1340 for determining the characteristics of the PI physiological parameter 1308. System 1330 may be similar or identical to system 1320 and/or system 1330 in some or many respects. With reference to FIG. 17, system 1340 may include a characteristic sub-system 1340a to record and/or identify trend characteristics 1341 and stability characteristics 1342 associated with the PI parameter 1306 which can be and a combination system 1330b calculating a weighed aggregate of the values from the parameter characteristics subsystem 1340a to determine the PI risk assessment 1354.

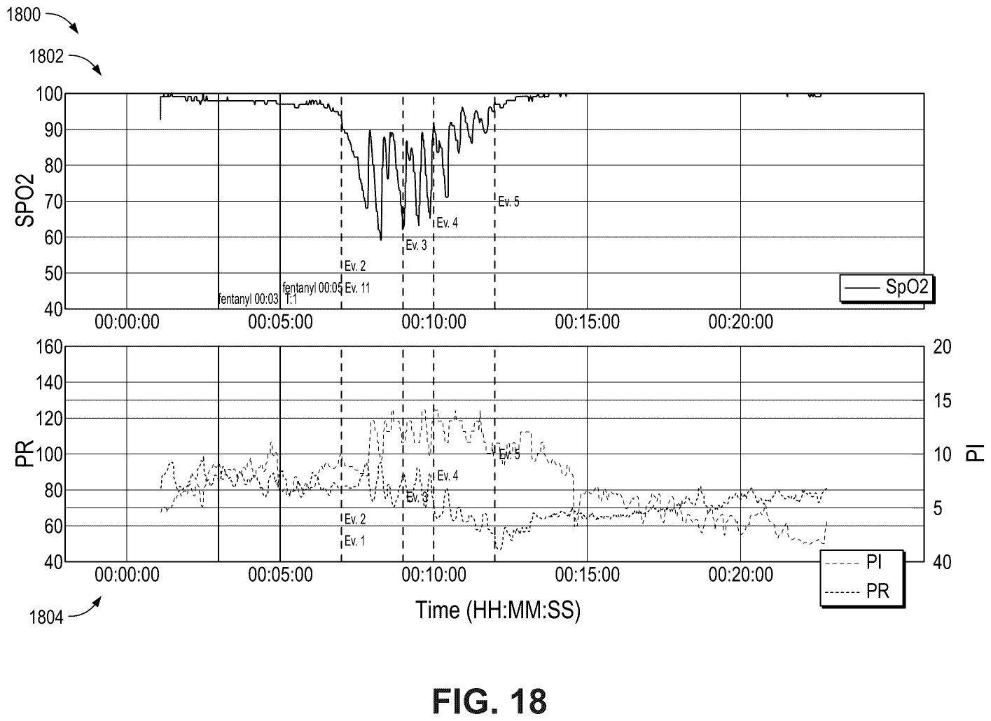

FIG. 18 illustrates example physiological data 1800 associated with an example opioid user's breathing cycle. As the user experiences an opioid overdose, the user's SpO₂ reflects the user's depressed respiration. For example, as SpO₂ decreases, an increased respiratory drive causes the user to perform a breakthrough breath, which increases the SpO₂ in the user's system. The user's SpO₂ can then decrease with an increased respiratory drive until the user performs another breakthrough breath. As shown in the graph 1802 illustrating the user's SpO₂, the SpO₂ can return to normal as the lack of SpO₂ is resolved with the breakthrough breaths. Also, as shown in graph 1804, the user's perfusion index and pulse rate can experience increased instability while the user is performing consecutive breakthrough breaths until the user returns to a normal breathing pattern.

### Terminology

The embodiments disclosed herein are presented by way of examples only and not to limit the scope of the claims that follow. One of ordinary skill in the art will appreciate from the disclosure herein that many variations and modifications can be realized without departing from the scope of the present disclosure.

The term “and/or” herein has its broadest least limiting meaning which is the disclosure includes A alone, B alone, both A and B together, or A or B alternatively, but does not require both A and B or require one of A or one of B. As used herein, the phrase “at least one of “A, B, “and” C should be construed to mean a logical A or B or C, using a non-exclusive logical or.

The description herein is merely illustrative in nature and is in no way intended to limit the disclosure, its application, or uses. For purposes of clarity, the same reference numbers will be used in the drawings to identify similar elements. It should be understood that steps within a method may be executed in different order without altering the principles of the present disclosure.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable components that provide the described functionality; or a combination of some or all of the above, such as in a system-on-chip. The term module may include memory (shared, dedicated, or group) that stores code executed by the processor.

The term code, as used above, may include software, firmware, and/or microcode, and may refer to programs, routines, functions, classes, and/or objects. The term shared, as used above, means that some or all code from multiple modules may be executed using a single (shared) processor. In addition, some or all code from multiple modules may be stored by a single (shared) memory. The term group, as used above, means that some or all code from a single module may be executed using a group of processors. In addition, some or all code from a single module may be stored using a group of memories.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage. Although the foregoing invention has been described in terms of certain preferred embodiments, other embodiments will be apparent to those of ordinary skill in the art from the disclosure herein. Additionally, other combinations, omissions, substitutions, and modifications will be apparent to the skilled artisan in view of the disclosure herein. Accordingly, the present invention is not intended to be limited by the reaction of the preferred embodiments, but is to be defined by reference to claims.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others.

Additionally, all publications, patents, and patent applications mentioned in this specification are herein incorporated by reference to the same extent as if each individual publication, patent, or patent application was specifically and individually indicated to be incorporated by reference.

1-51. (canceled)

52. An opioid overdose monitoring system configured to generate an overdose risk score of a user of a wearable device, the system comprising:
a physiological sensor coupled to the wearable device, said physiological sensor configured to detect attenuated light from a tissue site of the user;
one or more light emitting diodes of the physiological sensor configured to transmit an optical radiation into the tissue site of the user;
one or more detectors of the physiological sensor configured to respond to an intensity of the optical radiation after absorption by the tissue site of the user;
a display configured to display one or more screens; and
at least one hardware processor in communication with the physiological sensor, the at least one hardware processor configured to:
determine a plurality of parameters based at least on the attenuated light from the physiological sensor;
determine a plurality of characteristics based on the plurality of parameters, the plurality of characteristics associated with at least one of instantaneous values and historical values of the plurality of parameters, the plurality of characteristics comprising user trend characteristics and stability characteristics, wherein the user trend characteristics track the plurality of parameters over a period of time, and wherein the stability characteristics measure a degree of which instantaneous parameter values corresponding to the plurality of parameters tend to deviate from their baseline, wherein large and frequent deviations are indicative of high parameter instability;
determining an overdose risk score based on at least the user trend characteristics and the stability characteristics;
determine an alarm level of a series of escalating alarm levels based on the overdose risk score; and
implement an intervention associated with the determined alarm level.

53. The system of claim 52, wherein the plurality of parameters comprises at least one of oxygen saturation (SpO₂), respiration (PR), and perfusion index (PI).

54. The system of claim 53, wherein the plurality of parameters further comprises at one of respiration rate from the pleth (RRp) and temperature.

55. The system of claim 53, wherein the at least one hardware processor monitors a lower limit of the SpO₂.

56. The system of claim 52, wherein the at least one hardware processor is further configured to, for each of the plurality of parameters, determine a baseline risk, an instability index, an average slope, and desaturation pressure, and determine a weighted aggregate of the baseline risk, the instability index, the average slope, and the desaturation pressure.

57. The system of claim 52, wherein the alarm level is characterized by values of the overdose risk score, a normalized area corresponding to SpO₂ levels over a period of time, and SpO₂.

58. The system of claim 52, wherein the overdose risk score is at least based on a history of the plurality of parameter.

59. The system of claim 52, wherein the physiological sensor detects the plurality of parameters periodically.

60. The system of claim 52, wherein the at least one hardware processor is furthered configured to correlate one or more trends of the plurality of parameters.

61. The system of claim 60, wherein the at least one processor further correlates the trends of multiple physiological parameters.

62. The system of claim 52, wherein the at least one hardware processor is configured to determine a plurality of alarm levels in parallel.

63. The system of claim 52, wherein the at least one hardware processor is further configured to determines a presence of an event based on a crossing of at least one of a first and instantaneous baseline across one or more event thresholds.

64. The system of claim 52, wherein the alarm level is characterized by values of the overdose risk score, a normalized area, and a physiological parameter.

65. An opioid overdose monitoring system configured to generate an overdose risk score of a user, the system comprising:
a physiological sensor, said physiological sensor configured to detect attenuated light from a tissue site of the user;
one or more light emitting diodes of the physiological sensor configured to transmit an optical radiation into the tissue site of the user;
one or more detectors of the physiological sensor configured to respond to an intensity of the optical radiation after absorption by the tissue site of the user;
a display configured to display one or more screens; and
at least one hardware processor in communication with the physiological sensor, the at least one hardware processor configured to:
determine a plurality of parameters based on the attenuated light from the physiological sensor;
determine a plurality of characteristics based on the plurality of parameters, the plurality of characteristics associated with at least one of instantaneous values and historical values of the plurality of parameters, the plurality of characteristics comprising user trend characteristics and stability characteristics, wherein the user trend characteristics track the plurality of parameters over a period of time, and wherein the stability characteristics measure a degree of which instantaneous parameter values corresponding to the plurality of parameters tend to deviate from their baseline, where large and frequent deviations are indicative of high parameter instability;
determining an overdose risk score based on at least the user trend characteristics and the stability characteristics;
determine an alarm level of a series of escalating alarm levels based on the overdose risk score; and
implement an intervention associated with the determined alarm level.

66. The system of claim 65, wherein the plurality of parameters comprises at least one of oxygen saturation (SpO₂), respiration (PR), and perfusion index (PI).

67. The system of claim 66, wherein the plurality of parameters further comprises at one of respiration rate from the pleth (RRp) and temperature.

68. The system of claim 66, wherein the at least one hardware processor is further configured to, for each of the plurality of parameters, determine a baseline risk, an instability index, an average slope, and desaturation pressure, and determine a weighted aggregate of the baseline risk, the instability index, the average slope, and the desaturation pressure.

69. The system of claim 66, wherein the alarm level is characterized by values of the overdose risk score, a normalized area corresponding to SpO₂ levels over a period of time, and SpO₂.

70. The system of claim 66, wherein the overdose risk score is at least based on a history of the plurality of parameter.

71. The system of claim 66, wherein the at least one hardware processor is further configured to determine unavailability or unreliability of the plurality of parameters.
